(19) United States
(12) Patent Application Publication
(10) Pub. No.: US 2022/0039778 A1
(43) Pub. Date: Feb. 10, 2022

(54) DIAGNOSTIC ASSISTANCE DEVICE AND DIAGNOSTIC ASSISTANCE METHOD

(71) Applicants: TERUMO KABUSHIKI KAISHA, Tokyo (JP); Rokken Inc., Osaka (JP)

(72) Inventors: Yasukazu SAKAMOTO, Hiratsuka-shi (JP); Katsuhiko SHIMIZU, Fujinomiya-shi (JP); Hiroyuki ISHIHARA, Tokyo (JP); Itaru OKUBO, Naka-gun Ninomiya-cho (JP); Ryosuke SAGA, Osaka (JP); Thomas HENN, Osaka (JP); Clement JACQUET, Osaka (JP); Nuwan HERATH, Nancy (FR); Iselin ERIKSSEN, Osaka (JP)

(73) Assignees: TERUMO KABUSHIKI KAISHA, Tokyo (JP); Rokken Inc., Osaka (JP)

(21) Appl. No.: 17/510,531

(22) Filed: Oct. 26, 2021

Related U.S. Application Data
(63) Continuation of application No. PCT/JP2020/014319, filed on Mar. 27, 2020.

(30) Foreign Application Priority Data
Apr. 26, 2019 (JP) 2019-086061

(51) Int. Cl. A61B 8/08; G06T 15/20; A61B 8/12; A61B 8/00

(52) U.S. Cl. CPC A61B 8/5207; G06T 15/205; A61B 8/12; G06T 2210/41; A61B 8/483; A61B 8/4461; A61B 8/0891; A61B 8/54

(57) ABSTRACT

A diagnostic assistance device generates a three-dimensional image of a moving range of an ultrasound transducer from a two-dimensional image generated using the ultrasound transducer. The ultrasound transducer transmits ultrasound while moving inside a biological tissue through which blood passes. The diagnostic assistance device includes: a control unit that determines an upper limit of a third pixel number, the third pixel number being a pixel number in a third direction of the three-dimensional image corresponding to a moving direction of the ultrasound transducer, according to the number of the two-dimensional image generated per unit time, a first pixel number that is a pixel number in a first direction of the three-dimensional image corresponding to a horizontal direction of the two-dimensional image, and a second pixel number that is a pixel number in a second direction of the three-dimensional image corresponding to a vertical direction of the two-dimensional image.

### CROSS-REFERENCES TO RELATED APPLICATIONS

This application is a continuation of International Application No. PCT/JP2020/014319 filed on Mar. 27, 2020, which claims priority to Japanese Patent Application No. 2019-086061 filed on Apr. 26, 2019, the entire content of both of which is incorporated herein by reference.

### FIELD OF THE DISCLOSURE

The present disclosure generally relates to a diagnostic assistance device and a diagnostic assistance method.

### BACKGROUND DISCUSSION

U.S. Patent Application Publication No. 2010/0215238, U.S. Pat. Nos. 6,385,332, and 6,251,072 disclose a technique of generating a three-dimensional image of a cardiac cavity or a blood vessel using an ultrasound image system.

Treatment using intravascular ultrasound (IVUS) is widely performed on a cardiac cavity, a cardiac blood vessel, a lower limb artery region, and the like. The IVUS is a device or a method for providing a two-dimensional image of a plane perpendicular to a catheter major axis.

At present, an operator needs to perform treatment while reconstructing a three-dimensional structure by laminating two-dimensional images of IVUS in the head, which can be a barrier especially for a young doctor or an inexperienced doctor. In order to remove such a barrier, it is conceivable to automatically generate a three-dimensional image expressing a structure of a biological tissue such as a cardiac cavity or a blood vessel from the two-dimensional images of IVUS and display the generated three-dimensional image to the operator.

However, in order for the operator to perform treatment while referring to the three-dimensional image, it is necessary to generate the three-dimensional image in real time from the two-dimensional images of IVUS generated subsequent to a catheter operation. In the related art, it is only possible to create the three-dimensional image in the cardiac cavity or the blood vessel over time, and it is not possible to create the three-dimensional image in real time.

### SUMMARY

It would be desirable to limit a size of a three-dimensional space when a two-dimensional image of ultrasound is converted into a three-dimensional image to a size corresponding to the number of two-dimensional images generated per unit time.

A diagnostic assistance device according to an aspect of the disclosure generates a three-dimensional image of a moving range of an ultrasound transducer from a two-dimensional image generated using the ultrasound transducer. The ultrasound transducer transmits ultrasound while moving inside a biological tissue through which blood passes. The diagnostic assistance device includes: a control unit that determines an upper limit (Zm) of a third pixel number (Zn), the third pixel number (Zn) being a pixel number in a third direction of the three-dimensional image corresponding to a moving direction of the ultrasound transducer, according to the number (FPS) of the two-dimensional image generated per unit time, a first pixel number (Xn) that is a pixel number in a first direction of the three-dimensional image corresponding to a horizontal direction of the two-dimensional image, and a second pixel number (Yn) that is a pixel number in a second direction of the three-dimensional image corresponding to a vertical direction of the two-dimensional image.

As an embodiment of the disclosure, the control unit determines a product of a reference ratio (Xp or Yp) and a predetermined coefficient (α) as a setting ratio (Zp), the reference ratio (Xp or Yp) being a ratio of a dimension of the three-dimensional image in the first direction to the first pixel number (Xn) or a ratio of a dimension of the three-dimensional image in the second direction to the second pixel number (Yn), the setting ratio (Zp) being a ratio of a dimension of the three-dimensional image in the third direction to the third pixel number (Zn).

As an embodiment of the disclosure, the dimension of the three-dimensional image in the first direction is a horizontal dimension (Xd) of a range in which data on the two-dimensional image is acquired, and the dimension of the three-dimensional image in the second direction is a vertical dimension (Yd) of the range in which the data on the two-dimensional image is acquired.

As an embodiment of the disclosure, the ultrasound transducer moves in accordance with movement of a scanner unit, and the control unit sets a value obtained by dividing an upper limit (Mm) of a moving distance of the scanner unit by the product of the reference ratio (Xp or Yp) and the coefficient (α) as the third pixel number (Zn).

As an embodiment of the disclosure, the control unit warns a user if the value obtained by dividing the upper limit (Mm) of the moving distance of the scanner unit by the product of the reference ratio (Xp or Yp) and the coefficient (α) exceeds the upper limit (Zm) of the determined third pixel number (Zn).

As an embodiment of the disclosure, the control unit determines the product of the reference ratio (Xp or Yp) and the coefficient (α) as the setting ratio (Zp), and then determines a product of the reference ratio (Xp or Yp) and a coefficient (α′) after a change as a new setting ratio (Zp′) when the coefficient (α) is changed by a user.

As an embodiment of the disclosure, the ultrasound transducer moves in accordance with movement of a scanner unit, and when the coefficient (α) is changed by the user, if a value obtained by dividing an upper limit (Mm) of a moving distance of the scanner unit by the product of the reference ratio (Xp or Yp) and the coefficient (α′) after the change exceeds the upper limit (Zm) of the determined third pixel number (Zn), the control unit warns the user.

As an embodiment of the disclosure, the ultrasound transducer moves in accordance with movement of a scanner unit, and when the first pixel number (Xn) and the second pixel number (Yn) are changed by a user after the upper limit (Zm) of the third pixel number (Zn) is determined, the control unit warns the user if a value obtained by dividing an upper limit (Mm) of a moving distance of the scanner unit by a product of the coefficient (α) and a ratio of a dimension of the three-dimensional image in the first direction to a first pixel number (Xn′) after a change or a ratio of the dimension of the three-dimensional image in the second direction to a second pixel number (Yn′) after the change exceeds an upper limit (Zm′) of the third pixel number (Zn) corresponding to the number (FPS) of the two-dimensional image generated per unit time, the first pixel number (Xn′) after the change, and the second pixel number (Yn′) after the change.

As an embodiment of the disclosure, the control unit interpolates an image between generated two-dimensional images when a moving distance (Md) of the ultrasound transducer at each time interval at which the two-dimensional image is generated is larger than a product of the number (FPS) of the two-dimensional image generated per unit time and the determined setting ratio (Zp).

As an embodiment of the disclosure, the ultrasound transducer moves in accordance with movement of a scanner unit, and the control unit determines an interpolated image number by dividing a moving distance of the scanner unit at each time interval at which the two-dimensional image is generated by the determined setting ratio (Zp).

A diagnostic assistance method according to an aspect of the disclosure includes: transmitting, by an ultrasound transducer, ultrasound while moving inside a biological tissue through which blood passes; generating, by a diagnostic assistance device, a three-dimensional image of a moving range of the ultrasound transducer from a two-dimensional image generated by using the ultrasound transducer; and determining, by the diagnostic assistance device, an upper limit (Zm) of a third pixel number (Zn), the third pixel number (Zn) being a pixel number in a third direction of the three-dimensional image corresponding to a moving direction of the ultrasound transducer, according to the number (FPS) of the two-dimensional image generated per unit time, a first pixel number (Xn) that is a pixel number in a first direction of the three-dimensional image corresponding to a horizontal direction of the two-dimensional image, and a second pixel number (Yn) that is a pixel number in a second direction of the three-dimensional image corresponding to a vertical direction of the two-dimensional image.

In accordance with an aspect, a non-transitory computer readable medium (CRM) storing computer program code executed by a computer processor that executes a process for diagnostic assistance, the process comprising: generating a three-dimensional image of a moving range of an ultrasound transducer from a two-dimensional image generated by using the ultrasound transducer; and determining an upper limit of a third pixel number, according to the number of the two-dimensional image generated per unit time, a first pixel number, and a second pixel number, the first pixel number being a pixel number in a first direction of the three-dimensional image corresponding to a horizontal direction of the two-dimensional image, the second pixel number being a pixel number in a second direction of the three-dimensional image corresponding to a vertical direction of the two-dimensional image, the third pixel number being a pixel number in a third direction of the three-dimensional image corresponding to a moving direction of the ultrasound transducer.

According to the embodiments in the disclosure, it is possible to limit a size of a three-dimensional space when a two-dimensional image of ultrasound is converted into a three-dimensional image to a size corresponding to the number of two-dimensional images generated per unit time.

### BRIEF DESCRIPTION OF THE DRAWINGS

FIG. 1 is a perspective view of a diagnostic assistance system according to an embodiment of the disclosure.

FIG. 2 is a diagram showing a classification example of a plurality of pixels included in a two-dimensional image according to the embodiment of the disclosure.

FIG. 3 is a perspective view of a probe and a drive unit according to the embodiment of the disclosure.

FIG. 4 is a block diagram showing a configuration of a diagnostic assistance device according to the embodiment of the disclosure.

FIG. 5 is a flowchart showing an operation of the diagnostic assistance system according to the embodiment of the disclosure.

FIG. 6 is a diagram showing a data flow of the diagnostic assistance device according to the embodiment of the disclosure.

FIG. 7 is a diagram showing an input and output example of a learned model according to the embodiment of the disclosure.

FIG. 8 is a diagram showing a data flow of the diagnostic assistance device according to a modification of the embodiment of the disclosure.

FIG. 9 is a flowchart showing an operation of the diagnostic assistance device according to the embodiment of the disclosure.

FIG. 10 is a diagram showing a three-dimensional space according to the embodiment of the disclosure.

FIG. 11 is a flowchart showing an operation of the diagnostic assistance device according to the embodiment of the disclosure.

FIG. 12 is a flowchart showing an operation of the diagnostic assistance device according to the embodiment of the disclosure.

FIG. 13 is a flowchart showing an operation of the diagnostic assistance system according to the modification of the embodiment of the disclosure.

FIG. 14 is a diagram showing an example of an ultrasound maximum arrival range and a data acquisition range of ultrasound according to the embodiment of the disclosure.

### DETAILED DESCRIPTION

Set forth below with reference to the accompanying drawings is a detailed description of embodiments of a diagnostic assistance device and a diagnostic assistance method. Note that since embodiments described below are preferred specific examples of the present disclosure, although various technically preferable limitations are given, the scope of the present disclosure is not limited to the embodiments unless otherwise specified in the following descriptions.

In the drawings, the same or corresponding parts are denoted by the same reference numerals. In the description of the present embodiment, the description of the same or corresponding parts will be omitted or simplified as appropriate.

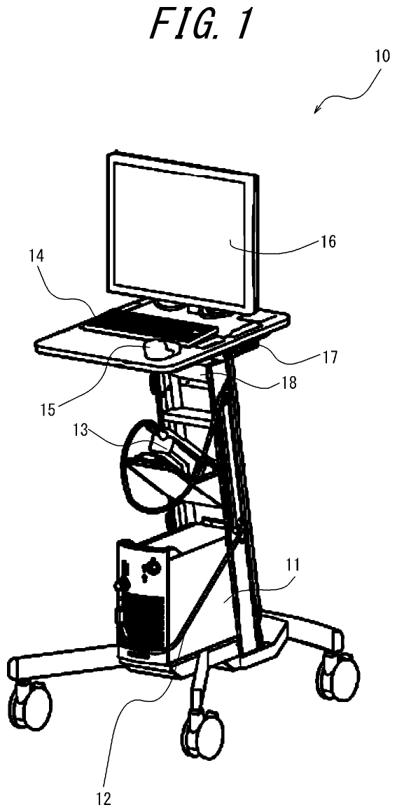

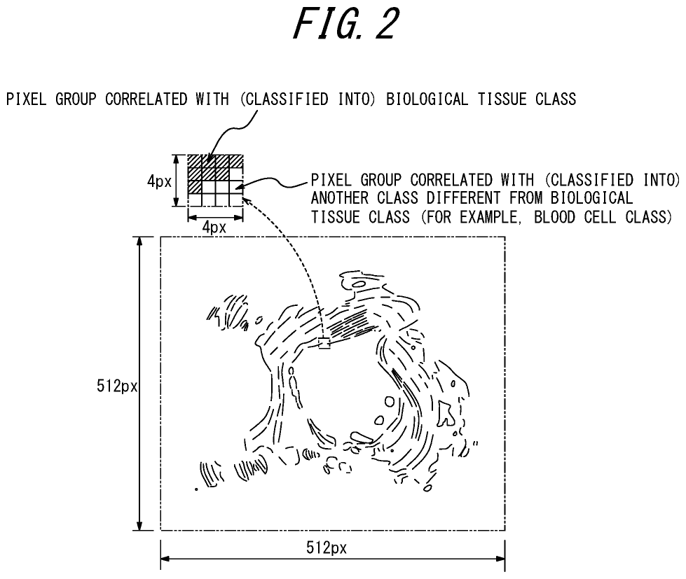

An outline of the present embodiment will be described with reference to FIGS. 1 and 2.

In the present embodiment, a diagnostic assistance device 11 correlates a plurality of pixels included in a two-dimensional image including a biological tissue, which is generated by processing a signal of a reflected wave of ultrasound transmitted inside the biological tissue through which blood passes, with two or more classes including a biological tissue class. The expression “correlates a plurality of pixels included in a two-dimensional image with classes” means that, in order to identify a type of an object such as the biological tissue displayed in each pixel of the two-dimensional image, a label such as a biological tissue label is given to each pixel, or that each pixel is classified into classes such as the biological tissue class. In the present embodiment, the diagnostic assistance device 11 generates a three-dimensional image of the biological tissue from a pixel group correlated with the biological tissue class. That is, the diagnostic assistance device 11 generates the three-dimensional image of the biological tissue from the pixel group classified into the biological tissue class. Then, a display 16 displays the three-dimensional image of the biological tissue generated by the diagnostic assistance device 11. In the example in FIG. 2, the plurality of pixels included in the two-dimensional image of 512 pixels×512 pixels (i.e., 512 pixels times 512 pixels), that is, 262,144 pixels are classified into two or more classes including the biological tissue class and another class such as a blood cell class. In a region of 4 pixels×4 pixels (i.e., 4 pixels times 4 pixels) displayed in an enlarged manner in FIG. 2, eight pixels, which are half of a total of 16 pixels, are a pixel group classified into the biological tissue class, and the remaining eight pixels are a pixel group classified into a class different from the biological tissue class. In FIG. 2, a pixel group of 4 pixels×4 pixels, which is a part of the plurality of pixels included in the two-dimensional image of 512 pixels×512 pixels, is displayed in an enlarged manner, and for convenience of description, the pixel group classified into the biological tissue class is hatched.

According to the present embodiment, accuracy of the three-dimensional image expressing the structure of the biological tissue, which is generated from the two-dimensional image of the ultrasound, can be improved.

In the present embodiment, an ultrasound transducer 25 transmits the ultrasound while moving inside the biological tissue through which the blood passes. The diagnostic assistance device 11 generates a three-dimensional image of a moving range of the ultrasound transducer 25 from two-dimensional images generated using the ultrasound transducer 25. The diagnostic assistance device 11 determines an upper limit Zm of a third pixel number Zn, which is a pixel number in a third direction of the three-dimensional image corresponding to a moving direction of the ultrasound transducer 25, according to the number FPS of two-dimensional images generated per unit time, a first pixel number Xn that is the pixel number in a first direction of the three-dimensional image corresponding to a horizontal direction of the two-dimensional images, and a second pixel number Yn that is the pixel number in a second direction of the three-dimensional image corresponding to a vertical direction of the two-dimensional images. The number FPS of the two-dimensional images generated per unit time can be represented by, for example, a frame rate, that is, the number of two-dimensional images generated per second.

According to the embodiment, a size of a three-dimensional space when a two-dimensional image of ultrasound is converted into a three-dimensional image can be limited to a size corresponding to the number of two-dimensional images generated per unit time.

In the present embodiment, the diagnostic assistance device 11 uses a two-dimensional image of IVUS as a two-dimensional image of ultrasound.

The IVUS can be used, for example, during intervention. Reasons for the use of the IVUS during intervention can be, for example, as follows: (1) to determine a biological tissue property in a cardiac cavity or the like; (2) to confirm a position for disposing an indwelling object such as a stent or a position at which the indwelling object is disposed; and (3) to confirm positions of a catheter other than an IVUS catheter and a guide wire using a two-dimensional image in real time.

Examples of the “catheter other than an IVUS catheter” described above can include a catheter for stent indwelling and an ablation catheter.

According to the present embodiment, an operator does not need to perform treatment while reconstructing a three-dimensional structure by laminating two-dimensional images of IVUS in the head. In particular, this makes the operation easier, especially for a younger doctor or an inexperienced doctor.

In the present embodiment, the diagnostic assistance device 11 can determine a positional relationship of a catheter other than an IVUS catheter, an indwelling object, or the like, or a biological tissue property in a three-dimensional image during surgery.

In the present embodiment, the diagnostic assistance device 11 can update the three-dimensional image in real time particularly in order to guide the IVUS catheter.

In a manipulation such as ablation, there is a demand for determining energy of ablation in consideration of a thickness of a blood vessel or a myocardial region. When an atherectomy device or the like for shaving calcified lesions or plaque is used, there is also a demand for performing a manipulation in consideration of a biological tissue thickness. In the present embodiment, the diagnostic assistance device 11 can display the thickness.

In the present embodiment, the diagnostic assistance device 11 continues updating the three-dimensional image by using an IVUS continuous image that is constantly updated, so that it is possible to continue providing a three-dimensional structure of a site that can be observed in a radial blood vessel manner.

In order to express a cardiac cavity structure based on the two-dimensional image of IVUS, it is necessary to distinguish a blood cell region, a myocardial region, a catheter other than the IVUS catheter in the cardiac cavity, and the like. In the present embodiment, the distinguishment is possible and the myocardial region alone can be displayed.

Since the IVUS uses a high frequency band of about 6 MHz to 60 MHz, blood cell noise can be greatly reflected, whereas in the present embodiment, it is possible to make a difference (or distinguish) between a biological tissue region and the blood cell region.

In order to perform in real time processing of expressing the cardiac cavity structure based on the two-dimensional image of IVUS updated, for example, at a speed of 15 frame per second (fps) or more and 90 fps or less (i.e., 15 fps to 90 fps), time for processing one image is limited to 11 msec or more and 66 msec or less (i.e., 11 msec to 66 msec). In the present embodiment, the diagnostic assistance device 11 can cope with the processing times as set forth above.

In the present embodiment, the diagnostic assistance device 11 can calculate a process of dropping an image (i.e., copying the image data (e.g. 2D image data) in three-dimensional space in order to display a three-dimensional image based on the image data) obtained by specifying the biological tissue property, removing the blood cell region, specifying a position of the catheter other than the IVUS catheter, or the like into a three-dimensional space and drawing a three-dimensional image until a next frame image comes, that is, within time in which a real time property is established.

In the present embodiment, the diagnostic assistance device 11 can provide not only a structure but also additional information in response to a request of a doctor, such as information on calcified lesions or plaque.

A configuration of a diagnostic assistance system 10 according to the present embodiment will be described with reference to FIG. 1.

The diagnostic assistance system 10 can include the diagnostic assistance device 11, a cable 12, a drive unit 13, a keyboard 14, a mouse 15, and the display 16.

The diagnostic assistance device 11 can be a dedicated computer specialized for image diagnosis in the present embodiment, and may be a general-purpose computer such as a personal computer (PC).

The cable 12 is used to connect the diagnostic assistance device 11 and the drive unit 13.

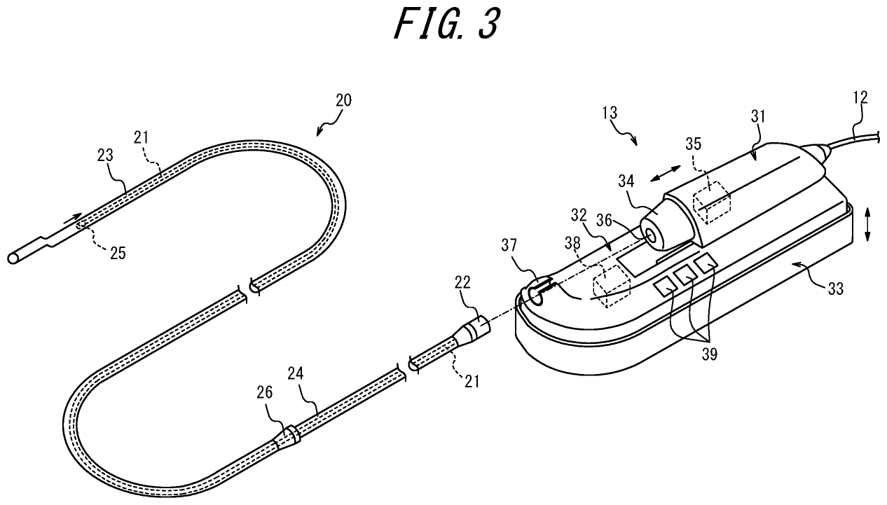

The drive unit 13 is a device that is connected to a probe 20 shown in FIG. 3 and drives the probe 20. The drive unit 13 is also referred to as a motor drive unit (MDU). The probe 20 is applied to an IVUS. The probe 20 is also referred to as an IVUS catheter or an image diagnosis catheter.

The keyboard 14, the mouse 15, and the display 16 can be connected to the diagnostic assistance device 11 via a cable or wirelessly. The display 16 can be, for example, a liquid crystal display (LCD), an organic electro luminescence (EL) display, or a head-mounted display (HMD).

The diagnostic assistance system 10 can further include a connection terminal 17 and a cart unit 18 as options.

The connection terminal 17 is used to connect the diagnostic assistance device 11 and an external device. The connection terminal 17 is, for example, a universal serial bus (USB) terminal. As the external device, for example, a recording medium such as a magnetic disk drive, a magneto-optical disk drive, or an optical disk drive can be used.

The cart unit 18 can be, for example, a cart with moving casters. The diagnostic assistance device 11, the cable 12, and the drive unit 13 are installed on a cart body of the cart unit 18. The keyboard 14, the mouse 15, and the display 16 are installed on an uppermost table of the cart unit 18.

A configuration of the probe 20 and the drive unit 13 according to the present embodiment will be described with reference to FIG. 3.

The probe 20 can include a drive shaft 21, a hub 22, a sheath 23, an outer tube 24, the ultrasound transducer 25, and a relay connector 26.

The drive shaft 21 passes through the sheath 23 to be inserted into a body-cavity of a living body and the outer tube 24 connected to a proximal end of the sheath 23, and extends to the inside of the hub 22 provided at a proximal end of the probe 20. The drive shaft 21 is provided with the ultrasound transducer 25 for transmitting and receiving a signal at a distal end of the drive shaft 21, and is rotatable in the sheath 23 and the outer tube 24. The relay connector 26 connects the sheath 23 and the outer tube 24.

The hub 22, the drive shaft 21, and the ultrasound transducer 25 are connected to each other to integrally move forward and backward in an axial direction. Therefore, for example, when the hub 22 is pressed toward a distal side, the drive shaft 21 and the ultrasound transducer 25 move inside the sheath 23 toward the distal side. For example, when the hub 22 is pulled toward a proximal side, the drive shaft 21 and the ultrasound transducer 25 move inside the sheath 23 toward the proximal side as indicated by arrows.

The drive unit 13 can include a scanner unit 31, a slide unit 32, and a bottom cover 33.

The scanner unit 31 is connected to the diagnostic assistance device 11 through the cable 12. The scanner unit 31 can include a probe connection unit 34 connected to the probe 20, and a scanner motor 35 that is a drive source for rotating the drive shaft 21.

The probe connection unit 34 can be freely and detachably connected to the probe 20 through an insertion port 36 of the hub 22 provided at the proximal end of the probe 20. The proximal end of the drive shaft 21 is rotatably supported inside the hub 22, and a rotational force of the scanner motor 35 is transmitted to the drive shaft 21. A signal is transmitted and received between the drive shaft 21 and the diagnostic assistance device 11 through the cable 12. In the diagnostic assistance device 11, a tomographic image of a body lumen is generated and image processing is performed based on the signal transmitted from the drive shaft 21.

The scanner unit 31 can be mounted on the slide unit 32 to be movable forward and backward, and is mechanically and electrically connected to the slide unit 32. The slide unit 32 can include a probe clamp unit 37, a slide motor 38, and a switch group 39.

The probe clamp unit 37 is disposed coaxially with the probe connection unit 34 on the distal side of the probe clamp unit 37, and supports the probe 20 connected to the probe connection unit 34.

The slide motor 38 is a drive source that generates a driving force in the axial direction. The scanner unit 31 moves forward and backward by the driving of the slide motor 38, and the drive shaft 21 accordingly moves forward and backward in the axial direction. The slide motor 38 can be, for example, a servo motor.

The switch group 39 can include, for example, a forward switch and a pull-back switch that are pressed when the scanner unit 31 is moved forward and backward, and a scan switch that is pressed when image drawing is started and ended. The disclosure is not limited to the above example, and various switches are included in the switch group 39 as necessary.

When the forward switch is pressed, the slide motor 38 rotates in a forward direction, and the scanner unit 31 moves forward. On the other hand, when the pull-back switch is pressed, the slide motor 38 rotates in a reverse direction, and the scanner unit 31 moves backward.

When the scan switch is pressed, the image drawing is started, the scanner motor 35 drives, and the slide motor 38 drives to move the scanner unit 31 backward. The operator connects the probe 20 to the scanner unit 31 in advance. The drive shaft 21 moves toward the proximal side in the axial direction while rotating when the image drawing starts. The scanner motor 35 and the slide motor 38 stop when the scan switch is pressed again, and the image drawing is ended.

The bottom cover 33 covers a bottom and an entire circumference of a side surface on a bottom side of the slide unit 32, and is movable toward and away from the bottom of the slide unit 32.

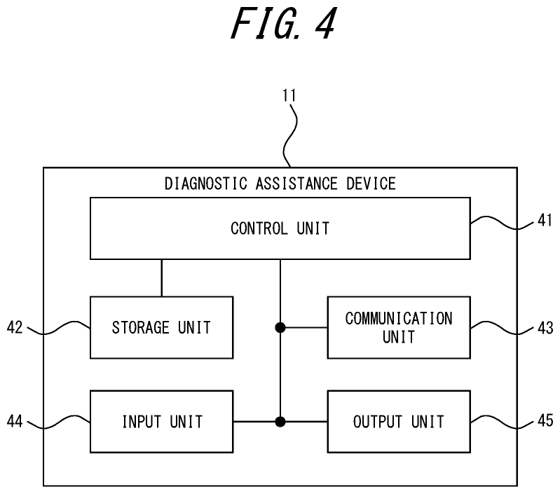

The configuration of the diagnostic assistance device 11 according to the present embodiment will be described with reference to FIG. 4.

The diagnostic assistance device 11 can include components such as a control unit 41, a storage unit 42, a communication unit 43, an input unit 44, and an output unit 45.

The control unit 41 can be one or more processors. As the processor, a general-purpose processor such as a central processing unit (CPU) or a graphics processing unit (GPU), or a dedicated processor specialized for specific processing can be used. The control unit 41 may include one or more dedicated circuits, or one or more processors in the control unit 41 may be replaced with one or more dedicated circuits. As the dedicated circuit, for example, a field-programmable gate array (FPGA) or an application specific integrated circuit (ASIC) can be used. The control unit 41 executes information processing related to the operation of the diagnostic assistance device 11 while controlling each unit of the diagnostic assistance system 10 including the diagnostic assistance device 11.

The storage unit 42 can be one or more memories. As the memory, for example, a semiconductor memory, a magnetic memory, or an optical memory can be used. As the semiconductor memory, for example, a random access memory (RAM) or a read only memory (ROM) can be used. As the RAM, for example, a static random access memory (SRAM) or a dynamic random access memory (DRAM) can be used. As the ROM, for example, an electrically erasable programmable read only memory (EEPROM) can be used. The memory functions as, for example, a main storage device, an auxiliary storage device, or a cache memory. The storage unit 42 stores information used for the operation of the diagnostic assistance device 11 and information obtained by the operation of the diagnostic assistance device 11.

The communication unit 43 is one or more communication interfaces. As the communication interface, a wired local area network (LAN) interface, a wireless LAN interface, or an image diagnosis interface for receiving and analog to digital (A/D) converting IVUS signals can be used. The communication unit 43 receives information used for the operation of the diagnostic assistance device 11 and transmits the information obtained by the operation of the diagnostic assistance device 11. In the present embodiment, the drive unit 13 is connected to an image diagnosis interface included in the communication unit 43.

The input unit 44 can be one or more input interfaces. As the input interface, for example, a USB interface or a high-definition multimedia interface HDMI® interface can be used. The input unit 44 receives an operation of inputting information used for the operation of the diagnostic assistance device 11. In the present embodiment, the keyboard 14 and the mouse 15 are connected to the USB interface included in the input unit 44. Alternatively, the keyboard 14 and the mouse 15 may be connected to the wireless LAN interface included in the communication unit 43.

The output unit 45 can be one or more output interfaces. As the output interface, for example, a USB interface or a HDMI interface can be used. The output unit 45 outputs the information obtained by the operation of the diagnostic assistance device 11. In the present embodiment, the display 16 is connected to the HDMI interface included in the output unit 45.

The function of the diagnostic assistance device 11 can be implemented by executing a diagnostic assistance program according to the present embodiment by a processor included in the control unit 41. That is, the function of the diagnostic assistance device 11 is implemented by software. The diagnostic assistance program is a program for causing a computer to execute processing of steps included in the operation of the diagnostic assistance device 11 and thereby implement a function corresponding to the processing of the steps. That is, the diagnostic assistance program is a program for causing the computer to function as the diagnostic assistance device 11.

The program can be recorded in a computer-readable recording medium.

As the computer-readable recording medium, for example, a magnetic recording device, an optical disk, a magneto-optical recording medium, or a semiconductor memory can be used. Distribution of the program is performed by, for example, selling, transferring, or lending a portable recording medium such as a digital versatile disc (DVD) or a compact disc read only memory (CD-ROM) on which the program is recorded. The program may be distributed by storing the program in a storage of a server and transferring the program from the server to another computer via a network. The program may be provided as a program product.

For example, the computer temporarily stores the program recorded in a portable recording medium or the program transferred from a server in the memory. Then, the computer causes the processor to read the program stored in the memory, and causes the processor to execute processing according to the read program. The computer may read the program directly from a portable recording medium and execute processing according to the program. Each time the program is transferred from the server to the computer, the computer may sequentially execute processing according to the received program. The processing may be executed by a so-called application service provider (ASP) type service in which the function is implemented by execution instruction and result acquisition alone without transferring the program from the server to the computer. The program includes information conforming to the program, which is information provided for processing by an electronic computer. For example, data that is not a direct command to the computer but has a property of defining the processing of the computer corresponds to the “information conforming to the program”.

A part or all of the functions of the diagnostic assistance device 11 may be implemented by the dedicated circuit included in the control unit 41. That is, a part or all of the functions of the diagnostic assistance device 11 may be implemented by the hardware.

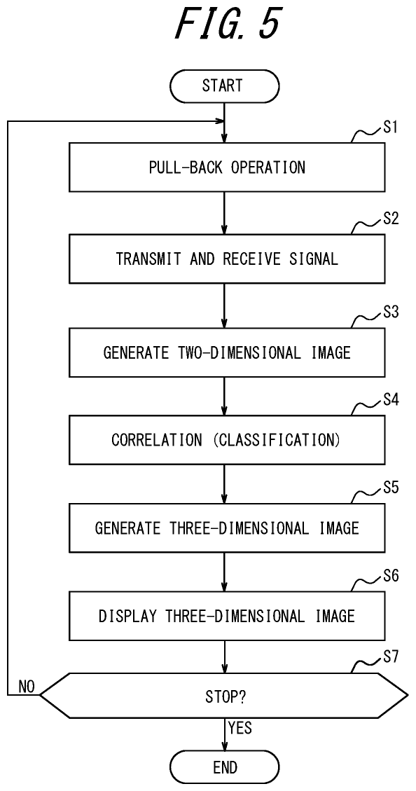

The operation of the diagnostic assistance system 10 according to the present embodiment will be described with reference to FIG. 5. The operation of the diagnostic assistance system 10 corresponds to a diagnostic assistance method according to the present embodiment.

Before the start of the flow in FIG. 5, the probe 20 is primed by the operator. Thereafter, the probe 20 is fitted into the probe connection unit 34 and the probe clamp unit 37 of the drive unit 13, and is connected and fixed to the drive unit 13. Then, the probe 20 is inserted to a target site in the biological tissue through which the blood passes, such as the cardiac cavity or the blood vessel.

In step S1, a so-called pull-back operation is performed by pressing the scan switch included in the switch group 39 and further pressing the pull-back switch included in the switch group 39. The probe 20 transmits, inside the biological tissue, the ultrasound by the ultrasound transducer 25 that moves backward in the axial direction by the pull-back operation.

In step S2, the probe 20 inputs a signal of a reflected wave of the ultrasound transmitted in step S1 to the control unit 41 of the diagnostic assistance device 11.

Specifically, the probe 20 transmits the signal of the ultrasound reflected inside the biological tissue to the diagnostic assistance device 11 through the drive unit 13 and the cable 12. The communication unit 43 of the diagnostic assistance device 11 receives the signal transmitted from the probe 20. The communication unit 43 performs A/D conversion on the received signal. The communication unit 43 inputs the A/D converted signal to the control unit 41.

In step S3, the control unit 41 of the diagnostic assistance device 11 processes the signal input in step S2 to generate a two-dimensional image of ultrasound.

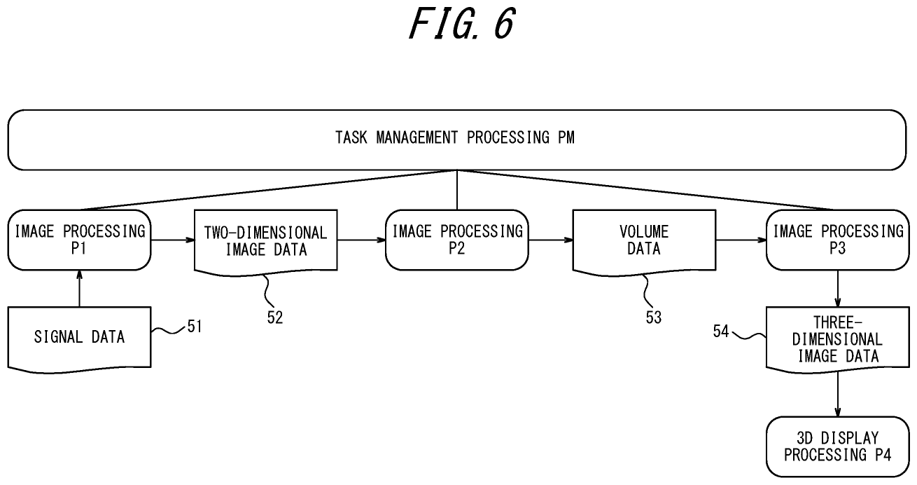

Specifically, as shown in FIG. 6, the control unit 41 executes task management processing PM for managing at least image processing P1, image processing P2, and image processing P3. A function of the task management processing PM is implemented, for example, as a function of an operating system (OS). The control unit 41 acquires the signal A/D converted by the communication unit 43 in step S2 as signal data 51. The control unit 41 activates the image processing P1 by the task management processing PM, processes the signal data 51, and generates the two-dimensional image of IVUS. The control unit 41 acquires the two-dimensional image of IVUS, which is a result of the image processing P1, as two-dimensional image data 52.

In step S4, the control unit 41 of the diagnostic assistance device 11 classifies a plurality of pixels included in the two-dimensional image generated in step S3 into two or more classes including the biological tissue class corresponding to pixels displaying the biological tissue. In the present embodiment, the two or more classes can further include a blood cell class corresponding to pixels displaying blood cells contained in blood. The two or more classes can further include a medical instrument class corresponding to pixels displaying a medical instrument such as a catheter other than the IVUS catheter or a guide wire. The two or more classes may further include an indwelling object class corresponding to pixels displaying an indwelling object, for example, such as a stent. The two or more classes may further include a lesion class corresponding to pixel displaying a lesion such as calcified lesions or plaque. Each class may be subdivided. For example, the medical instrument class may be divided into a catheter class, a guide wire class, and other medical instrument classes.

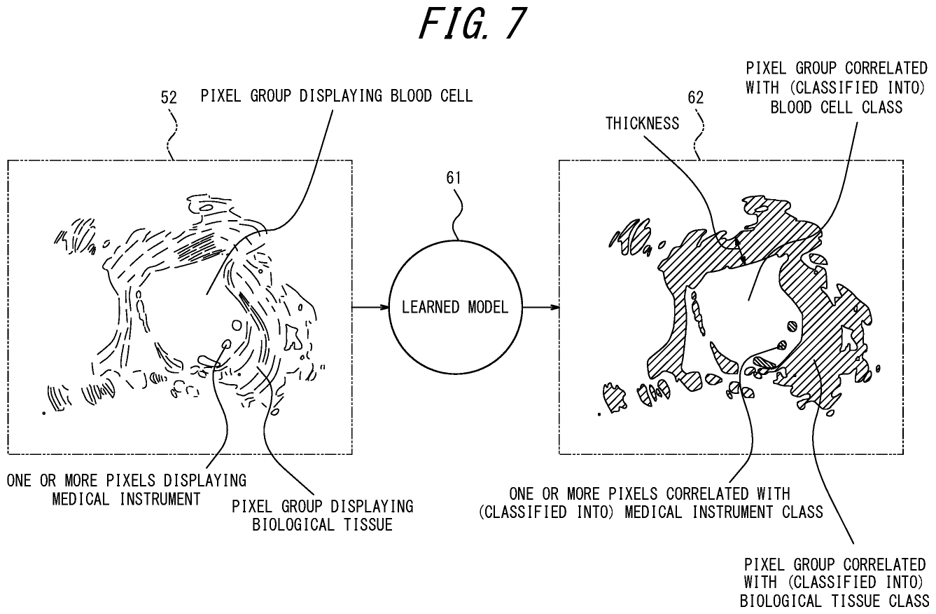

Specifically, as shown in FIGS. 6 and 7, the control unit 41 activates the image processing P2 by the task management processing PM, and classifies the plurality of pixels included in the two-dimensional image data 52 acquired in step S3 using a learned model 61. The control unit 41 acquires, as a classification result 62, two-dimensional images obtained by classifying the pixels of the two-dimensional image data 52 that is the result of the image processing P2 into the biological tissue class, the blood cell class, and the medical instrument class.

In step S5, the control unit 41 of the diagnostic assistance device 11 generates a three-dimensional image of the biological tissue from a pixel group classified into the biological tissue class in step S4. In the present embodiment, the control unit 41 generates the three-dimensional image of the biological tissue by excluding a pixel group classified into the blood cell class in step S4 from the plurality of pixels included in the two-dimensional image generated in step S3. The control unit 41 generates a three-dimensional image of the medical instrument from one or more pixels classified into the medical instrument class in step S4. Furthermore, when two or more pixels displaying different medical instruments are included in the one or more pixels classified into the medical instrument class in step S4, the control unit 41 generates the three-dimensional image of the medical instrument for each medical instrument.

Specifically, as shown in FIG. 6, the control unit 41 executes the image processing P2 by the task management processing PM, laminates the two-dimensional images in which the pixels of the two-dimensional image data 52 acquired in step S4 are classified, and converts the two-dimensional images into the three-dimensional image. The control unit 41 acquires volume data 53 expressing a three-dimensional structure for each classification, which is a result of the image processing P2. Then, the control unit 41 activates the image processing P3 by the task management processing PM, and visualizes the acquired volume data 53. The control unit 41 acquires, as three-dimensional image data 54, a three-dimensional image expressing the three-dimensional structure for each classification, which is a result of the image processing P3.

As a modification of the present embodiment, the control unit 41 may generate the three-dimensional image of the medical instrument based on coordinates of one or more pixels classified into the medical instrument class in step S4. Specifically, the control unit 41 may hold data indicating the coordinates of the one or more pixels classified into the medical instrument class in step S4 as coordinates of a plurality of points present along the moving direction of the scanner unit 31 of the drive unit 13, and generate a linear three-dimensional model connecting the plurality of points along the moving direction of the scanner unit 31 as the three-dimensional image of the medical instrument. For example, for a medical instrument having a small cross section such as the catheter, the control unit 41 may dispose the three-dimensional model having a circular cross section as the three-dimensional image of the medical instrument at coordinates at a center of one pixel classified into the medical instrument class or a center of a pixel group classified into the medical instrument class. That is, in the case of a small object, for example, such as the catheter, the coordinates may be returned as the classification result 62 instead of a pixel or a region that is a set of pixels.

In step S6, the control unit 41 of the diagnostic assistance device 11 performs control to display the three-dimensional image of the biological tissue generated in step S5. In the present embodiment, the control unit 41 performs control to display the three-dimensional image of the biological tissue and the three-dimensional image of the medical instrument generated in step S5 in a format distinguishable from each other. When the three-dimensional image of the medical instrument is generated for each medical instrument in step S5, the control unit 41 performs control to display the generated three-dimensional image of the medical instrument in the format distinguishable for each medical instrument. The display 16 displays the three-dimensional image of the biological tissue and the three-dimensional image of the medical instrument under the control of the control unit 41.

Specifically, as shown in FIG. 6, the control unit 41 executes 3D display processing P4, and displays the three-dimensional image data 54 acquired in step S6 on the display 16 through the output unit 45. The three-dimensional image of the biological tissue such as the cardiac cavity or the blood vessel and the three-dimensional image of the medical instrument such as the catheter are displayed in a distinguishable manner by being applied with different colors or the like. In accordance with an exemplary embodiment, either of the three-dimensional image of the biological tissue and the three-dimensional image of the medical instrument may be selected by the keyboard 14 or the mouse 15. In this case, the control unit 41 receives an operation of selecting the image through the input unit 44. The control unit 41 can display the selected image on the display 16 through the output unit 45, and can hide the unselected image. Any cut cross section may be set by the keyboard 14 or the mouse 15. In this case, the control unit 41 receives the operation of selecting a cut cross section through the input unit 44. The control unit 41 displays the three-dimensional image cut by the selected cut cross section on the display 16 through the output unit 45.

In step S7, when the scan switch included in the switch group 39 is not pressed again, the processing returns to step S1 and the pull-back operation is continued. As a result, the two-dimensional images of IVUS are sequentially generated while changing a transmission position of the ultrasound inside the biological tissue. On the other hand, when the scan switch is pressed again, the pull-back operation is stopped, and the flow in FIG. 5 ends.

In the present embodiment, the image processing P1 and the 3D display processing P4 can be executed on the CPU, and the image processing P2 and the image processing P3 can be executed on the GPU. The volume data 53 may be stored in a storage region in the CPU, but is stored in a storage region in the GPU in order to omit data transfer between the CPU and the GPU.

In particular, the classification, catheter detection, image interpolation, and three-dimensional conversion included in the image processing P2 are executed in the general purpose graphics processing unit (GP-GPU) in the present embodiment, and may be executed in an integrated circuit such as a FPGA or an ASIC. The processing may be executed in series or in parallel. Each processing may be executed via a network.

In step S4, the control unit 41 of the diagnostic assistance device 11 extracts the biological tissue region by region recognition instead of edge extraction in the related art. Reasons for the extraction of the biological tissue with region by region recognition will be described.

In an IVUS image, the three-dimensional image can be created by extracting an edge indicating a boundary between the blood cell region and the biological tissue region for a purpose of removing the blood cell region and reflecting the edge in a three-dimensional space. However, the edge extraction has a fairly high degree of difficulty in the following points:
A brightness gradient at the boundary between the blood cell region and the biological tissue region is not constant, and it can be difficult to solve all edges with a uniform algorithm.
When creating the three-dimensional image with the edge, a complicated structure cannot be expressed, for example, when targeting not only a blood vessel wall but also an entire cardiac cavity.
In an image in which the blood cell region is included not only inside the biological tissue but also outside the biological tissue, such as a portion where both a left atrium and a right atrium can be seen, the edge extraction alone is not sufficient.
It is not possible to specify a catheter by extracting the edge alone. In particular, when a wall of the biological tissue is in contact with the catheter, it is not possible to take a boundary between the biological tissue and the catheter.
When a thin wall is sandwiched, it is difficult to know which side is a real biological tissue by the edge alone.
It can be difficult to calculate the thickness.

In steps S2 to S6, when performing three-dimensional conversion, the control unit 41 of the diagnostic assistance device 11 needs to remove blood cell components, extract an organ portion, reflect information on the organ portion in the three-dimensional space, and draw the three-dimensional image. The processing can be completed within time Tx when an image is sent to continuously update the three-dimensional image in real time. The time Tx is 1/FPS. In the related art, for example, for providing the three-dimensional image, real time processing cannot be implemented. In the method in the related art, the processing is performed for each frame, and a three-dimensional image cannot be continuously updated until the next frame comes.

As described above, in the present embodiment, each time a new two-dimensional image is generated, the control unit 41 generates a three-dimensional image of the biological tissue corresponding to the newly generated two-dimensional image before the next two-dimensional image is generated.

Specifically, the control unit 41 generates the two-dimensional image of IVUS at a speed of 15 times or more per second and 90 times or less per second (i.e., 15 times per second to 90 times per second), and updates the three-dimensional image at a speed of 15 times or more per second and 90 times or less per second (i.e., 15 times per second to 90 times per second).

In step S4, the control unit 41 of the diagnostic assistance device 11 can specify a particularly small object such as the catheter by extracting a region of an object other than the biological tissue by the region recognition instead of the edge extraction as in the related art, and thus can cope with the following problems, which can include when the catheter is in contact with a wall, even a person determines that the catheter is a biological tissue from only one image, and by mistaking the catheter with a thrombus or a bubble, it can be difficult to determine the catheter with only one image.

The control unit 41 may use past information to specify the catheter position as in a case where a human ordinary estimates the catheter position using a past continuous image as reference information.

In step S4, even when a body of the probe 20 at the center of the two-dimensional image and a wall surface are in contact with each other, the control unit 41 of the diagnostic assistance device 11 can distinguish the body from the wall surface by extracting a region of an object other than the biological tissue by the region recognition instead of the edge extraction as in the related art. That is, the control unit 41 can separate the IVUS catheter itself from the biological tissue region.

In step S4, the control unit 41 of the diagnostic assistance device 11 extracts the biological tissue region and a catheter region instead of the edge extraction in order to express a complicated structure, determine the biological tissue property, and search for the small object, for example, such as the catheter. Therefore, in the present embodiment, an approach of machine learning can be adopted. Using the learned model 61, the control unit 41 directly evaluates a property of a portion for each pixel of the image, and reflects the classified image in a three-dimensional space set under a predetermined condition. The control unit 41 laminates the information in the three-dimensional space, performs the three-dimensional conversion based on the information stored in a three-dimensionally disposed memory space, and displays the three-dimensional image. The processing is updated in real time, and three-dimensional information at the position corresponding to the two-dimensional image is updated. In accordance with an exemplary embodiment, calculation can be performed sequentially or in parallel. In particular, temporal efficiency is improved by performing the processing in parallel.

The machine learning refers to analyzing input data using an algorithm, extracting a useful rule, a determination criterion, or the like from an analysis result, and developing the algorithm. The algorithm of the machine learning is generally classified into supervised learning, unsupervised learning, reinforcement learning, and the like. In the supervised learning algorithm, a data set of input of sound data and an ultrasound image of biological sound that is a sample and a result of data of a disease corresponding to the sound data and the ultrasound image is given, and the machine learning is performed based on the data set. In the unsupervised learning algorithm, only a large amount of input data is given and the machine learning is performed. In the reinforcement learning algorithm, environment is changed based on a solution output by the algorithm, and correction is made based on a reward indicating how correct the output solution is. The machine-learned model thus obtained is used as the learned model 61.

In accordance with an exemplary embodiment, the learned model 61 is trained such that a class can be specified from the two-dimensional image that is the sample by performing the machine learning in advance. The ultrasound image that is the sample and an image obtained by performing classification in which a person labels the ultrasound image in advance are collected in, for example, a medical institution such as a university hospital in which many patients gather.

The IVUS image can include high noise such as the blood cell region, and can further include system noise. Therefore, in step S4, the control unit 41 of the diagnostic assistance device 11 performs preprocessing on the image before inserting the image into the learned model 61. As the preprocessing, for example, smoothing using various filters such as simple blur, median blur, Gaussian blur, bilateral filter, median filter, and block averaging, or image morphology such as dilation and erosion, opening and closing, morphological gradient, or top hat and black hat, or flood fill, resize, image pyramids, threshold, low path filter, high path filter, or discrete wavelet transform can be performed. However, when such processing is performed on a normal CPU, the processing alone may not be completed within, for example, 66 msec. Therefore, the processing is performed on the GPU. In particular, in an approach with the machine learning constructed by a plurality of layers called deep learning, it has been verified that it is possible to perform the preprocessing with the real time property by constructing an algorithm as the layer. In the verification, an image of 512 pixels×512 pixels or more is used to achieve classification accuracy, for example, of 97% or more and 42 fps.

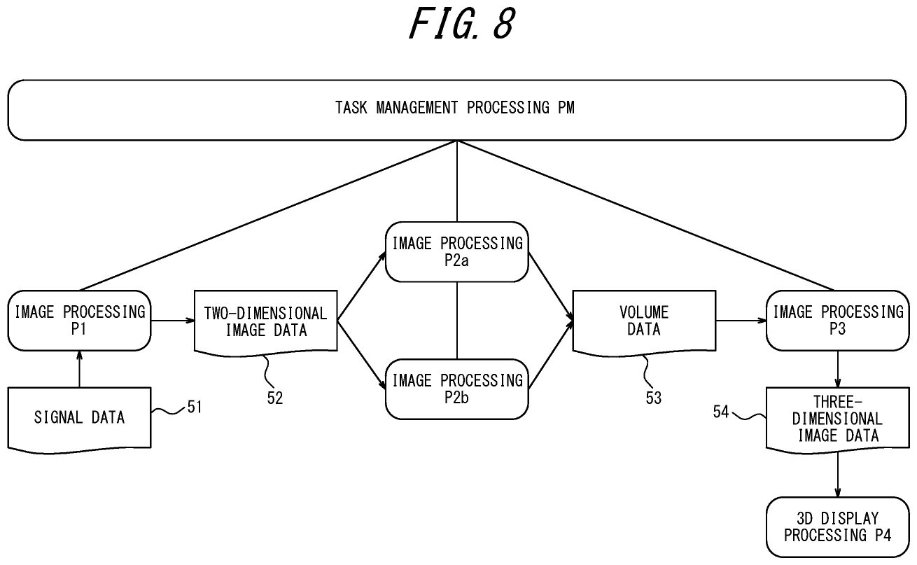

When cases with and without the preprocessing are compared, it is desirable to add the layer of the preprocessing in the extraction of the biological tissue region, whereas when a small object such as the catheter in the two-dimensional image is determined, it is preferable that there is no layer of the preprocessing. Therefore, as a modification of the present embodiment, different image processing P2 may be prepared for each class. For example, as shown in FIG. 8, image processing P2a including the layer of the preprocessing for the biological tissue class and image processing P2b not including the layer of the preprocessing for the catheter class or for specifying the catheter position may be prepared.

In the modification, the control unit 41 of the diagnostic assistance device 11 smoothes the two-dimensional image. The smoothing processing is processing of smoothing shading variation of the pixel group. The smoothing processing includes the smoothing described above. The control unit 41 executes first classification processing of classifying a plurality of pixels included in the two-dimensional image before being smoothed into the medical instrument class and one or more other classes. The control unit 41 executes second classification processing of classifying the pixel group included in the smoothed two-dimensional image into one or more classes including the biological tissue class, excluding one or more pixels classified into the medical instrument class in the first classification processing. The control unit 41 superimposes one or more pixels classified in the first classification processing and the pixel group classified in the second classification processing, thereby displaying the medical instrument on the three-dimensional image with high accuracy. As a further modification of the modification, the control unit 41 may execute the first classification processing of classifying the plurality of pixels included in the two-dimensional image before being smoothed into the medical instrument class and one or more other classes, and the second classification processing of smoothing the two-dimensional image excluding the one or more pixels classified into the medical instrument class in the first classification processing and classifying the pixel group included in the smoothed two-dimensional image into one or more classes including the biological tissue class.

In step S5, the control unit 41 of the diagnostic assistance device 11 measures the biological tissue thickness using biological tissue region information acquired as a result of the classification by the image processing P2. The control unit 41 expresses the thickness by reflecting a measurement result in the three-dimensional information. In step S6, the control unit 41 displays the thickness by performing processing such as dividing the three-dimensional structure by coloring using gradation or the like. The control unit 41 may further provide additional information by a display method such as changing the color of the three-dimensional biological tissue structure for each class, such as a difference in the biological tissue property.

As described above, in the present embodiment, the control unit 41 calculates the biological tissue thickness by analyzing the pixel group classified into the biological tissue class in step S4. The control unit 41 performs control to display the calculated biological tissue thickness. The display 16 is controlled by the control unit 41 to display the biological tissue thickness. As a modification of the present embodiment, the control unit 41 may calculate the biological tissue thickness by analyzing the generated three-dimensional image of the biological tissue.

A definition of the three-dimensional space in the present embodiment will be described.

As a method of three-dimensional conversion, a rendering method such as surface rendering or volume rendering, and various operations such as texture mapping, bump mapping, and environment mapping associated with the rendering method can be used.

In accordance with an exemplary embodiment, the three-dimensional space used in the present embodiment is limited to a size in which the real time processing can be performed. The size is required to be based on the FPS for acquiring the ultrasound image defined in the system.

In the present embodiment, the drive unit 13 capable of acquiring the position one by one is used. The scanner unit 31 of the drive unit 13 can move on a single axis, and the axis is defined as a z axis, and a position of the scanner unit 31 at a certain moment is defined as z. The z axis is associated with one axis in a predetermined three-dimensional space, and the axis is defined as a Z axis. Since the Z axis and the z axis are linked, a point Z on the Z axis is predetermined so that Z=f(z).

Information on the classification result 62 obtained by the image processing P2 is reflected on the Z axis. In an XY axis plane of the three-dimensional space defined here, it is required that all class information that can be classified by the image processing P2 can be stored. Furthermore, it is desirable that luminance information in an original ultrasound image is included at the same time. In the information on the classification result 62 obtained by the image processing P2, all the class information is reflected on an XY plane at a three-dimensional Z axis position corresponding to a current position of the scanner unit 31.

Although it is desirable that the three-dimensional space is three-dimensionally converted by using volume rendering or the like for each Tx (=1/FPS), the three-dimensional space cannot be increased infinitely since processing time is limited. That is, the three-dimensional space is required to have a size that can be calculated within Tx (=1/FPS).

When it is desired to convert a long range on the drive unit 13 into three dimensions, the long range may not fall within a calculable size. Therefore, Z=f(z) is defined as an appropriate conversion in order to keep the range displayed by the drive unit 13 within the above described range. This means that it is necessary to set a function for converting the position on the Z axis into the position on the z axis within both limits of the moving range of the scanner unit 31 of the drive unit 13 on the Z axis and the range in which the volume data 53 can be saved on the z axis.

As described above, in the present embodiment, the control unit 41 of the diagnostic assistance device 11 classifies the plurality of pixels included in the two-dimensional image generated by processing the signal of the reflected wave of the ultrasound transmitted inside the biological tissue through which the blood passes into two or more classes including the biological tissue class corresponding to the pixels displaying the biological tissue. The control unit 41 generates the three-dimensional image of the biological tissue from the pixel group classified into the biological tissue class. The control unit 41 performs control to display the generated three-dimensional image of the biological tissue. Therefore, according to the present embodiment, the accuracy of the three-dimensional image expressing the structure of the biological tissue generated from the two-dimensional image of the ultrasound can be improved.

According to the present embodiment, the three-dimensional image is displayed in real time, the operator can perform the manipulation without converting the two-dimensional images into the three-dimensional space in the head, and it is expected to reduce the fatigue of the operator and shorten the manipulation time.

According to the present embodiment, a positional relationship of an inserted object such as the catheter or the indwelling object such as the stent, for example, can be clear, and thus failure of the manipulation is reduced.

According to the present embodiment, the property of the biological tissue can be three-dimensionally grasped, and an accurate manipulation can be performed.

According to the present embodiment, the accuracy can be improved by inserting the layer of the preprocessing into the image processing P2.

According to the present embodiment, the biological tissue thickness can be measured using the classified biological tissue region information, and the information can be reflected in the three-dimensional information.

In the present embodiment, an ultrasound image is used as an input image, and the classification is performed by classifying an output for each pixel or a region in which the plurality of pixels are regarded as a set into two or more classes, including a catheter body region, a blood cell region, a calcified region, a fibrosis region, a catheter region, a stent region, a myocardial necrosis region, a fat biological tissue, or a biological tissue between organs, thereby it is possible to determine which portion is which region in one image.

In the present embodiment, the classification of the biological tissue class corresponding to at least the heart and the blood vessel region is determined in advance. Learning efficiency can be improved by using, as a material of the machine learning, supervised data, in which each pixel or a region in which the plurality of pixels are regarded as a set is already classified into two or more classes including the biological tissue class.

In the present embodiment, the learned model 61 is constructed as any neural network for deep learning including convolutional neural network (CNN), recurrent neural network (RNN), and long short-term memory (LSTM).

As a modification of the present embodiment, instead of the diagnostic assistance device 11 performing the processing in step S3, another device may perform the processing in step S3, and the diagnostic assistance device 11 may acquire the two-dimensional image generated as a result of the processing in step S3 and perform the processing in step S4 and subsequent steps. That is, instead of the control unit 41 of the diagnostic assistance device 11 processing the IVUS signal to generate the two-dimensional image, another device may process the IVUS signal to generate the two-dimensional image and input the generated two-dimensional image to the control unit 41.

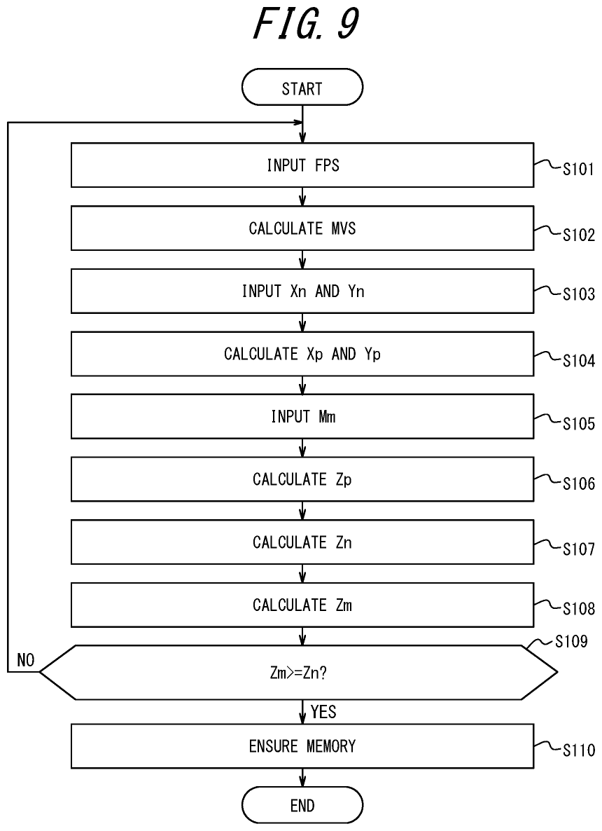

With reference to FIG. 9, an operation of setting the size of the three-dimensional space in order for the diagnostic assistance device 11 to generate the three-dimensional image in real time based on the two-dimensional images of IVUS sequentially generated in accordance with the catheter operation by the operator will be described. The operation is performed before the operation in FIG. 5.

In step S101, the control unit 41 receives, through the input unit 44, an operation of inputting the number FPS of two-dimensional images generated per unit time by processing the signal of the reflected wave of the ultrasound from the ultrasound transducer 25 that transmits the ultrasound while moving inside the biological tissue through which the blood passes.

Specifically, the control unit 41 displays on the display 16 a screen for selecting the number FPS of the two-dimensional images of IVUS generated per unit time or specifically specifying the number FPS through the output unit 45. On the screen for selecting the number FPS of the two-dimensional images of IVUS generated per unit time, options such as 30 fps, 60 fps, and 90 fps are displayed. The control unit 41 acquires, through the input unit 44, a numerical value of the number FPS of the two-dimensional images of IVUS generated per unit time, which is selected or specified by a user such as the operator using the keyboard 14 or the mouse 15. The control unit 41 stores the acquired numerical value of the number FPS of the two-dimensional images of IVUS generated per unit time in the storage unit 42.

As a modification of the present embodiment, the numerical value of the number FPS of the two-dimensional images of IVUS generated per unit time may be stored in advance in the storage unit 42.

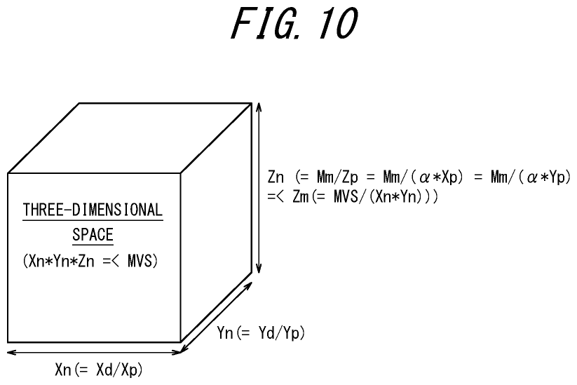

In step S102, the control unit 41 determines a maximum volume size MVS in the three-dimensional space according to the number FPS of the two-dimensional images generated per unit time input in step S101. In accordance with an exemplary embodiment, it is assumed that the maximum volume size MVS of the three-dimensional space is determined in advance or calculated for each numerical value or numerical value range of a candidate of the number FPS of the two-dimensional images generated per unit time, depending on specifications of the computer that is the diagnostic assistance device 11. As shown in FIG. 10, the size of the three-dimensional space is a product of the first pixel number Xn that is the pixel number in the first direction of the three-dimensional image corresponding to the horizontal direction of the two-dimensional image, the second pixel number Yn that is the pixel number in the second direction of the three-dimensional image corresponding to the vertical direction of the two-dimensional image, and the third pixel number Zn that is the pixel number in the third direction of the three-dimensional image corresponding to the moving direction of the ultrasound transducer 25. At this time, all of the first pixel number Xn, the second pixel number Yn, and the third pixel number Zn are undetermined. In the present embodiment, the horizontal direction of the two-dimensional image is an X direction, the vertical direction of the two-dimensional image is a Y direction, and the order may be reversed. In the present embodiment, the first direction of the three-dimensional image is the X direction, the second direction of the three-dimensional image is the Y direction, and the third direction of the three-dimensional image is the Z direction, and the order of the X direction and the Y direction may be reversed.

In accordance with an exemplary embodiment, the control unit 41 calculates the maximum volume size MVS of the three-dimensional space that corresponds to the numerical value of the number FPS of the two-dimensional images generated per unit time that is stored in the storage unit 42 in step S101 using a conversion table stored in the storage unit 42 in advance or a predetermined calculation formula. The control unit 41 stores the numerical value of the calculated maximum volume size MVS in the storage unit 42.

Here, a Voxel value theoretical value calculation method based on a transfer rate will be described.

Data transfer from the CPU to the GPU can be performed, for example, through PCI Express®. For example, 1 GB/s is used as a standard, and the transfer rate is determined by a multiple of 1 GB/s. In the PCI Express installed in many cases, the GPU uses ×16 in many cases. Here, it is determined that 16 GB can be transferred per ×16, that is, per second.

In view of the specification of the system, if the screen is updated at 15 fps or more and 30 fps or less (i.e., 15 fps to 30 fps), the transfer between the CPU and the GPU per time need to be performed at 1/30 [fps]=0.033 [fps]. In consideration of this, an amount of Voxel data that can be theoretically transferred is 16 GB/s×0.033=0.533 GB=533 MB, which is an upper limit of a transfer size. A data size changes depending on how a Voxel unit is expressed. Here, if each Voxel is expressed by 8 bits, that is, if each Voxel is represented by 0 to 255, a size of about 512×512×2000 can be handled.

However, in practice, the size cannot be used for processing. Specifically, when calculation time required to update the data is considered, it is considered that X fps is guaranteed when the following expression is satisfied.

1/X [fps]>=Tf(S)+Tp(V)+F(V)

The formula is a type of processing time before and after transfer. Here, Tf(S) is time of a filter required for processing a pixel of a size S (=X×Y), Tp(V) is processing time required for creating Voxel and preparing for transfer, and F(V) is transfer time and drawing time of Voxel of size V (=X×Y×Z). Tp(V) is small enough to be ignored. If a processing speed of the filter is f [fps] (where X<=f), a theoretically transferable upper limit value can be calculated by the following calculation formula.

Voxel size<=16 GB×(1/X−1/f−F(V))

For example, when X=15 and F=30, it is possible to spend 0.033 seconds for other processing such as volume rendering and transfer time. If the time can be devoted only to the transfer, it is theoretically possible to transfer the Voxel with the upper limit of 512×512×8138, that is, the maximum volume size MVS.

In step S103, the control unit 41 receives an operation of inputting the first pixel number Xn and the second pixel number Yn through the input unit 44. The first pixel number Xn and the second pixel number Yn may be different numbers, but are the same number in the present embodiment.

Specifically, the control unit 41 displays on the display 16 a screen for selecting the first pixel number Xn and the second pixel number Yn or specifically specifying the first pixel number Xn and the second pixel number Yn through the output unit 45. On the screen for selecting the first pixel number Xn and the second pixel number Yn, for example, options such as 512×512 and 1024×1024 are displayed. The control unit 41 acquires numerical values of the first pixel number Xn and the second pixel number Yn selected or specified by the user using the keyboard 14 or the mouse 15 through the input unit 44. The control unit 41 stores the acquired numerical values of the first pixel number Xn and the second pixel number Yn in the storage unit 42.

As a modification of the present embodiment, the numerical values of the first pixel number Xn and the second pixel number Yn may be stored in advance in the storage unit 42.

In step S104, the control unit 41 calculates a reference ratio Xp that is a ratio of a dimension of the three-dimensional image in the first direction to the first pixel number Xn input in step S103. Alternatively, the control unit 41 calculates a reference ratio Yp that is a ratio of the dimension of the three-dimensional image in the second direction to the second pixel number Yn input in step S103. The dimension of the three-dimensional image in the first direction is a horizontal dimension Xd of a range in which the data of the two-dimensional image is acquired. The dimension of the three-dimensional image in the second direction is a vertical dimension Yd of the range in which data of the two-dimensional image is acquired. Each of the horizontal dimension Xd and the vertical dimension Yd is a physical distance in the biological tissue in an actual space. The physical distance in the biological tissue in the actual space is calculated from the speed and time of the ultrasound. That is, the dimension of the three-dimensional image in the first direction is an actual dimension in the horizontal direction of the range expressed by the three-dimensional image in the living body. The dimension in the second direction of the three-dimensional image is an actual dimension in the vertical direction of the range expressed by the three-dimensional image in the living body. The range expressed by the three-dimensional image in the living body may include not only the biological tissue but also a peripheral portion of the biological tissue. The horizontal dimension Xd of the range in which the data of the two-dimensional image is acquired and the vertical dimension Yd of the range in which the data of the two-dimensional image is acquired can also be input by the user by estimating the physical distance in the biological tissue.

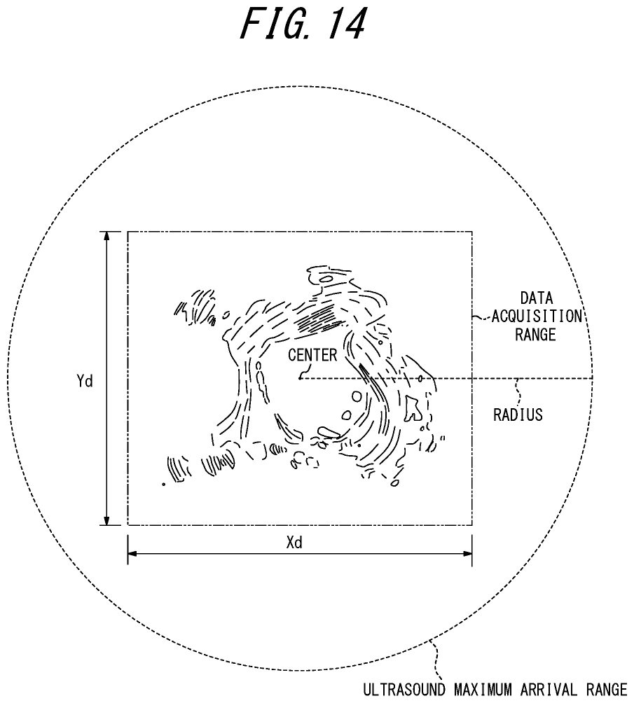

Specifically, the control unit 41 acquires the numerical value of the horizontal dimension Xd of the data acquisition range of IVUS stored in advance in the storage unit 42. The control unit 41 divides the obtained numerical value of the horizontal dimension Xd by the numerical value of the first pixel number Xn stored in the storage unit 42 in step S103 to obtain the reference ratio Xp. That is, the control unit 41 calculates Xp=Xd/Xn. The control unit 41 stores the obtained reference ratio Xp in the storage unit 42. Alternatively, the control unit 41 acquires the numerical value of the vertical dimension Yd of the data acquisition range of IVUS stored in advance in the storage unit 42. The control unit 41 divides the obtained numerical value of the vertical dimension Yd by the numerical value of the second pixel number Yn stored in the storage unit 42 in step S103 to obtain the reference ratio Yp. That is, the control unit 41 calculates Yp=Yd/Yn. The control unit 41 stores the obtained reference ratio Yp in the storage unit 42. As shown in FIG. 14, the ultrasound maximum arrival range of IVUS is a maximum range in which the two-dimensional image can be generated from the reflected waves of the ultrasound reflected by the biological tissue. In the present embodiment, since the three-dimensional image is displayed in real time, the ultrasound maximum arrival range is a circle having a radius equal to a distance obtained by multiplying 1/“predetermined FPS” by the speed of the ultrasound. The data acquisition range of IVUS is a range acquired as data of the two-dimensional image. The data acquisition range can be arbitrarily set as the whole or a part of the ultrasound maximum arrival range. In accordance with an exemplary embodiment, the horizontal dimension Xd and the vertical dimension Yd of the data acquisition range are both equal to or less than a diameter of the ultrasound maximum arrival range. For example, when the radius of the ultrasound maximum arrival range is 80 mm, each of the horizontal dimension Xd and the vertical dimension Yd of the data acquisition range is set to any value larger than 0 mm and equal to or smaller than the diameter 160 mm of the ultrasound maximum arrival range. Since the horizontal dimension Xd and the vertical dimension Yd of the data acquisition range are physical distances in the biological tissue in the actual space, values of the horizontal dimension Xd and the vertical dimension Yd are determined, and even if the three-dimensional image is enlarged or reduced, the reference ratio Xp and the reference ratio Yp do not change.

In step S105, the control unit 41 receives an operation of inputting an upper limit Mm of a moving distance of the scanner unit 31 through the input unit 44. The ultrasound transducer 25 moves along with the movement of the scanner unit 31, and the moving distance of the ultrasound transducer 25 coincides with the moving distance of the scanner unit 31. In the present embodiment, the moving distance of the scanner unit 31 is a distance by which the scanner unit 31 moves backward by the pull-back operation.

Specifically, the control unit 41 displays on the display 16 a screen for selecting the upper limit Mm of the moving distance of the scanner unit 31 or specifically specifying the upper limit Mm through the output unit 45. On the screen for selecting the upper limit Mm, for example, options such as 15 cm, 30 cm, 45 cm, and 60 cm can be displayed. The control unit 41 acquires the upper limit Mm of the moving distance of the scanner unit 31 selected or specified by the user using the keyboard 14 or the mouse 15 through the input unit 44. The control unit 41 stores the acquired upper limit Mm in the storage unit 42.

As a modification of the present embodiment, the upper limit Mm of the moving distance of the scanner unit 31 may be stored in the storage unit 42 in advance.

In step S106, the control unit 41 determines a product of the reference ratio Xp or the reference ratio Yp calculated in step S104 and a predetermined coefficient α as a setting ratio Zp that is a ratio of the dimension of the three-dimensional image in the third direction to the third pixel number Zn. The coefficient α can be, for example, 1.0. The dimension of the three-dimensional image in the third direction is a dimension in the moving direction of a range in which the ultrasound transducer 25 moves. That is, the dimension of the three-dimensional image in the third direction is an actual dimension in a depth direction of the range expressed by the three-dimensional image in the living body. The dimension in the moving direction is a physical distance in the biological tissue in the actual space. Therefore, even if the three-dimensional image is enlarged or reduced, the setting ratio Zp does not change.

Specifically, the control unit 41 multiplies the reference ratio Xp or the reference ratio Yp stored in the storage unit 42 in step S104 by the coefficient α stored in advance in the storage unit 42 to obtain the setting ratio Zp. That is, the control unit 41 calculates Zp=α×Xp or Zp=α×Yp. The control unit 41 stores the obtained setting ratio Zp in the storage unit 42.

In step S107, the control unit 41 determines, as the third pixel number Zn, a value obtained by dividing the upper limit Mm of the moving distance of the scanner unit 31 input in step S105 by the setting ratio Zp determined in step S106. This is to match the upper limit Mm of the moving distance of the scanner unit 31 with the actual dimension of all the pixels of the three-dimensional image in the third direction.

Specifically, the control unit 41 divides the upper limit Mm of the moving distance of the scanner unit 31 stored in the storage unit 42 in step S105 by the setting ratio Zp stored in the storage unit 42 in step S106 to obtain the third pixel number Zn. That is, the control unit 41 calculates Zn=Mm/Zp. The control unit 41 stores the obtained numerical value of the third pixel number Zn in the storage unit 42.

In step S108, the control unit 41 determines, as the upper limit Zm of the third pixel number Zn, a value obtained by dividing the maximum volume size MVS of the three-dimensional space determined in step S102 by the product of the first pixel number Xn and the second pixel number Yn input in step S103.

Specifically, the control unit 41 obtains the upper limit Zm of the third pixel number Zn by dividing the numerical value of the maximum volume size MVS stored in the storage unit 42 in step S102 by the product of the numerical value of the first pixel number Xn and the numerical value of the second pixel number Yn stored in the storage unit 42 in step S103. That is, the control unit 41 calculates Zm=MVS/(Xn×Yn). The control unit 41 stores the obtained upper limit Zm in the storage unit 42.

In step S109, the control unit 41 compares the third pixel number Zn determined in step S107 with the upper limit Zm of the third pixel number Zn determined in step S108.

Specifically, the control unit 41 determines whether the numerical value of the third pixel number Zn stored in the storage unit 42 in step S107 exceeds the upper limit Zm stored in the storage unit 42 in step S108.

If the third pixel number Zn exceeds the upper limit Zm, the processing returns to step S101 and resetting is performed. In the resetting, in order to implement the real time processing, the control unit 41 notifies the user through the output unit 45 that it is necessary to change at least one of the number FPS of the two-dimensional images generated per unit time input in step S101, the first pixel number Xn and the second pixel number Yn input in step S103, and the upper limit Mm of the moving distance of the scanner unit 31 input in step S105. That is, the control unit 41 warns the user.

When the third pixel number Zn is equal to or less than the upper limit Zm, the processing moves to step S110, and the memory can be ensured. In the present embodiment, the memory is a storage region of the volume data 53 that is an entity of the three-dimensional space, and specifically, is a storage region in the GPU.

As described above, in the present embodiment, the diagnostic assistance device 11 generates the three-dimensional image of the moving range of the ultrasound transducer 25 from the two-dimensional images generated using the ultrasound transducer 25 that transmits the ultrasound while moving inside the biological tissue through which the blood passes. The control unit 41 of the diagnostic assistance device 11 determines the upper limit Zm of the third pixel number Zn, which is the pixel number in the third direction of the three-dimensional image corresponding to the moving direction of the ultrasound transducer 25, according to the number FPS of the two-dimensional images generated per unit time, the first pixel number Xn that is the pixel number in the first direction of the three-dimensional image corresponding to the horizontal direction of the two-dimensional image, and the second pixel number Yn that is the pixel number in the second direction of the three-dimensional image corresponding to the vertical direction of the two-dimensional image. Therefore, according to the present embodiment, it is possible to limit the size of the three-dimensional space when the two-dimensional images of ultrasound are converted into the three-dimensional image to the size corresponding to the number of two-dimensional images generated per unit time.

According to the present embodiment, the size of the three-dimensional space can be limited to or smaller than a size capable of generating the three-dimensional image in real time from the two-dimensional images of IVUS sequentially generated in accordance with the catheter operation. As a result, the operator can perform treatment while referring to the three-dimensional image.

In accordance with an exemplary embodiment, an actual scale of one pixel of the two-dimensional image of IVUS is a predetermined fixed value. The fixed value is referred to as “depth”. Since the three-dimensional conversion must be performed at a size that can be calculated within 1/FPS, a maximum number of volume pixels is determined by the FPS specifically. Therefore, when the pixel numbers on the X axis, the Y axis, and the Z axis in the three-dimensional space are Xn, Yn, and Zn, respectively, a relationship of Xn×Yn×Zn=<MVS can be established. When the actual scales per pixel of the X axis, the Y axis, and the Z axis are Xp, Yp, and Zp, respectively, Xp=Yp=depth/(Xn or Yn) and Zp=α×Xp=α×Yp are satisfied. Although α is basically 1, when the three-dimensional image is actually constructed, the three-dimensional image that is not suitable for the image of the operator may be completed. In such a case, it is possible to construct the three-dimensional image close to a clinical cardiac cavity or a blood vessel image by adjusting a.

The actual scale to be three-dimensionally converted may be automatically determined from the relationship of Xn, Yn, depth, and FPS, whereas when the operator tries to set a pull-back distance, Zn corresponding to the distance may exceed Zm. In this case, Xn, Yn, Zn, Xp, Yp, and Zp need to be modified again.

In this manner, each scale of one pixel along the X axis, the Y axis, and the Z axis is correlated with the actual distance in reality, so that it is possible to construct the three-dimensional image with more reality. By separately providing the a value, it is possible to construct an actual image of the inside of the cardiac cavity imaged by the doctor. According to the present embodiment, it is possible to construct the three-dimensional image imitating the actual scale while updating the three-dimensional image in real time.

As described below, in the present embodiment, for example, the user can adjust the coefficient α.

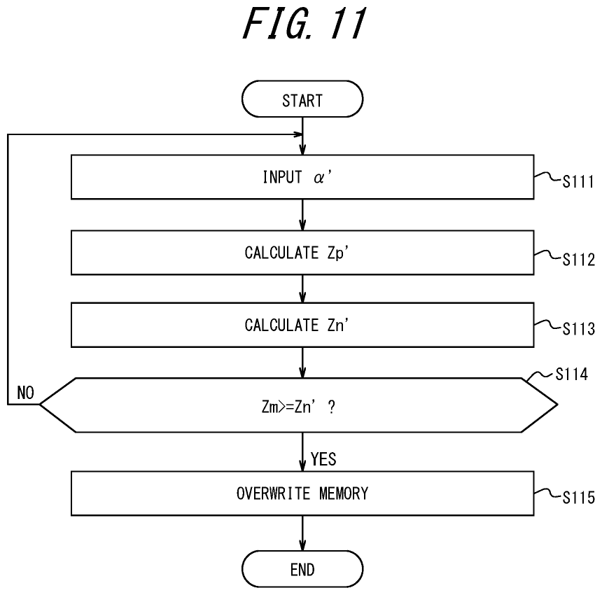

With reference to FIG. 11, the operation of the diagnostic assistance device 11 when the coefficient α is changed by the user after the diagnostic assistance device 11 determines the product of the reference ratio Xp and the coefficient α as the setting ratio Zp in step S106 will be described. The operation may be performed before the operation in FIG. 5, or may be performed during or after the operation in FIG. 5.

In step S111, the control unit 41 receives an operation of inputting a changed coefficient α′ through the input unit 44.

Specifically, the control unit 41 displays, on the display 16 through the output unit 45, a screen for selecting a value of the coefficient α′ after the change or specifically specifying the value of the coefficient α′ while indicating a current value of the coefficient α. The control unit 41 acquires, through the input unit 44, the coefficient α′ after the change selected or specified by the user such as the operator using the keyboard 14 or the mouse 15. The control unit 41 stores the acquired coefficient α′ in the storage unit 42.

In step S112, the control unit 41 determines the product of the reference ratio Xp or the reference ratio Yp calculated in step S104 and the coefficient α′ after the change input in step S111 as a new setting ratio Zp′.

Specifically, the control unit 41 multiplies the reference ratio Xp or the reference ratio Yp stored in the storage unit 42 in step S104 by the coefficient α′ stored in the storage unit 42 in step S111 to obtain the setting ratio Zp′. That is, the control unit 41 calculates Zp′=α′×Xp or Zp′=α′×Yp. The control unit 41 stores the obtained setting ratio Zp′ in the storage unit 42.

In step S113, the control unit 41 determines, as the third pixel number Zn′, a value obtained by dividing the upper limit Mm of the moving distance of the scanner unit 31 input in step S105 by the setting ratio Zp′ determined in step S112.

Specifically, the control unit 41 divides the upper limit Mm of the moving distance of the scanner unit 31 stored in the storage unit 42 in step S105 by the setting ratio Zp′ stored in the storage unit 42 in step S112 to obtain the third pixel number Zn′. That is, the control unit 41 calculates Zn′=Mm/Zp′. The control unit 41 stores the obtained numerical value of the third pixel number Zn′ in the storage unit 42.

In step S114, the control unit 41 compares the third pixel number Zn′ determined in step S113 with the upper limit Zm of the third pixel number Zn determined in step S108.

Specifically, the control unit 41 determines whether the numerical value of the third pixel number Zn′ stored in the storage unit 42 in step S113 exceeds the upper limit Zm stored in the storage unit 42 in step S108.

If the third pixel number Zn′ exceeds the upper limit Zm, the processing returns to step S111 and resetting is performed. In the resetting, in order to implement the real time processing, the control unit 41 notifies the user through the output unit 45 that it is necessary to cancel the change of the coefficient α in step S111 or to change the coefficient α to a value different from the coefficient α′ in step S111. That is, the control unit 41 warns the user. As a modification, the control unit 41 may notify the user that it is necessary to change at least one of the number FPS of the two-dimensional images generated per unit time, the first pixel number Xn, the second pixel number Yn, and the upper limit Mm of the moving distance of the scanner unit 31 while adopting the coefficient α′ after the change.

When the third pixel number Zn′ is equal to or less than the upper limit Zm, the processing moves to step S115, and the image data in the three-dimensional space is overwritten by the latest image data.

According to the present embodiment, after the three-dimensional image is actually constructed, the coefficient α is modified, and the doctor who is the operator can correct a three-dimensional scale in order to bring the three-dimensional image close to an actual image. In accordance with an exemplary embodiment, the user may have an unnatural feeling, for example, when α=1.0, and a three-dimensional image closer to the image can be constructed by adjusting the coefficient α.

As described below, in the present embodiment, the user can adjust the first pixel number Xn and the second pixel number Yn.

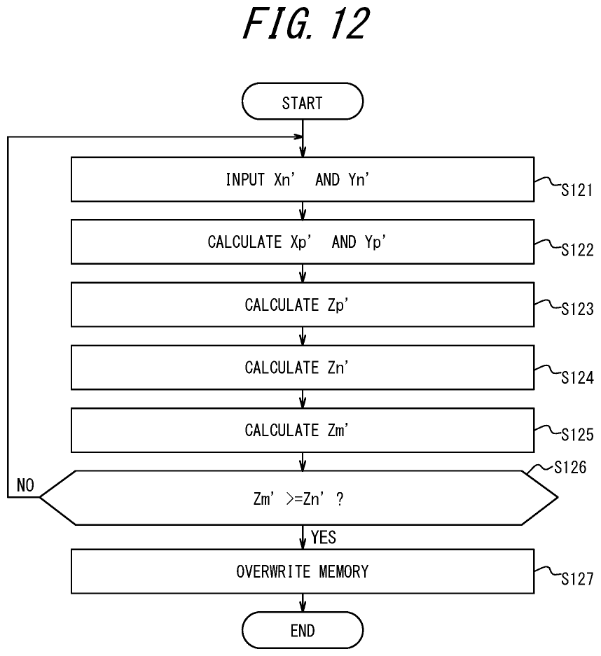

With reference to FIG. 12, an operation of the diagnostic assistance device 11 when the first pixel number Xn and the second pixel number Yn can be changed by the user after the diagnostic assistance device 11 determines the upper limit Zm of the third pixel number Zn in step S107 will be described. The operation may be performed before the operation in FIG. 5, or may be performed during or after the operation in FIG. 5.

In step S121, the control unit 41 receives an operation of inputting the changed first pixel number Xn′ and the changed second pixel number Yn′ through the input unit 44. The first pixel number Xn′ and the second pixel number Yn′ after the change may be different numbers, but are the same number in the present embodiment.

Specifically, the control unit 41 displays, on the display 16 through the output unit 45, a screen for selecting the first pixel number Xn′ and the second pixel number Yn′ after the change or specifically specifying the first pixel number Xn′ and the second pixel number Yn′ while indicating current values of the first pixel number Xn and the second pixel number Yn. The control unit 41 acquires the numerical values of the first pixel number Xn′ and the second pixel number Yn′ after the change selected or specified by the user using the keyboard 14 or the mouse 15 through the input unit 44. The control unit 41 stores the acquired numerical values of the first pixel number Xn′ and the second pixel number Yn′ in the storage unit 42.

In step S122, the control unit 41 calculates a reference ratio Xp′ that is a ratio of the dimension of the three-dimensional image in the first direction to a changed first pixel number Xn′ input in step S121. Alternatively, the control unit 41 calculates a reference ratio Yp′ that is a ratio of the dimension of the three-dimensional image in the second direction to a changed second pixel number Yn′ input in step S121.

Specifically, the control unit 41 acquires the numerical value of the horizontal dimension Xd of the data acquisition range of IVUS stored in advance in the storage unit 42. The control unit 41 divides the obtained numerical value of the horizontal dimension Xd by the numerical value of the first pixel number Xn stored in the storage unit 42 in step S121 to obtain the reference ratio Xp′. That is, the control unit 41 calculates Xp′=Xd/Xn′. The control unit 41 stores the obtained reference ratio Xp′ in the storage unit 42. Alternatively, the control unit 41 acquires the numerical value of the vertical dimension Yd of the data acquisition range of IVUS stored in advance in the storage unit 42. The control unit 41 divides the obtained numerical value of the vertical dimension Yd by the numerical value of the second pixel number Yn′ stored in the storage unit 42 in step S103 to obtain the reference ratio Yp′. That is, the control unit 41 calculates Yp′=Yd/Yn′. The control unit 41 stores the obtained reference ratio Yp′ in the storage unit 42.

In step S123, the control unit 41 determines the product of the reference ratio Xp′ or the reference ratio Yp′ calculated in step S122 and the coefficient α as a new setting ratio Zp′.

Specifically, the control unit 41 multiplies the reference ratio Xp′ or the reference ratio Yp′ stored in the storage unit 42 in step S122 by the coefficient α stored in advance in the storage unit 42 to obtain the setting ratio Zp′. That is, the control unit 41 calculates Zp′=α×Xp′ or Zp′=α×Yp′. The control unit 41 stores the obtained setting ratio Zp′ in the storage unit 42.

In step S124, the control unit 41 determines, as the third pixel number Zn′, a value obtained by dividing the upper limit Mm of the moving distance of the scanner unit 31 input in step S105 by the setting ratio Zp′ determined in step S123.

Specifically, the control unit 41 divides the upper limit Mm of the moving distance of the scanner unit 31 stored in the storage unit 42 in step S105 by the setting ratio Zp′ stored in the storage unit 42 in step S123 to obtain the third pixel number Zn′. That is, the control unit 41 calculates Zn′=Mm/Zp′. The control unit 41 stores the obtained numerical value of the third pixel number Zn′ in the storage unit 42.

In step S125, the control unit 41 determines, as the upper limit Zm′ of the third pixel number Zn′, a value obtained by dividing the maximum volume size MVS of the three-dimensional space determined in step S102 by the product of the first pixel number Xn′ and the second pixel number Yn′ after the change input in step S121.

Specifically, the control unit 41 obtains the upper limit Zm′ of the third pixel number Zn′ by dividing the numerical value of the maximum volume size MVS stored in the storage unit 42 in step S102 by the product of the numerical value of the first pixel number Xn′ and the numerical value of the second pixel number Yn′ stored in the storage unit 42 in step S121. That is, the control unit 41 calculates Zm′=MVS/(Xn′×Yn′). The control unit 41 stores the obtained upper limit Zm′ in the storage unit 42.

In step S126, the control unit 41 compares the third pixel number Zn′ determined in step S124 with the upper limit Zm′ of the third pixel number Zn′ determined in step S125.

Specifically, the control unit 41 determines whether the numerical value of the third pixel number Zn′ stored in the storage unit 42 in step S124 exceeds the upper limit Zm′ stored in the storage unit 42 in step S125.

If the third pixel number Zn′ exceeds the upper limit Zm′, the processing returns to step S121 and resetting is performed. In the resetting, in order to implement the real time processing, the control unit 41 notifies the user through the output unit 45 that it is necessary to cancel the change of the first pixel number Xn and the second pixel number Yn in step S121 or to change the first pixel number Xn and the second pixel number Yn to values different from the first pixel number Xn′ and the second pixel number Yn′ in step S121. That is, the control unit 41 warns the user. As a modification, the control unit 41 may notify the user that it is necessary to change at least one of the number FPS of the two-dimensional images generated per unit time, the coefficient α, and the upper limit Mm of the moving distance of the scanner unit 31 while adopting the changed first pixel number Xn′ and the changed second pixel number Yn′.

When the third pixel number Zn′ is equal to or less than the upper limit Zm′, the processing moves to step S127, and the memory is overwritten.

In accordance with an exemplary embodiment, the actual scale of one pixel of the two-dimensional image of IVUS can be a predetermined fixed value. The fixed value is referred to as “depth”. Since the three-dimensional conversion needs to be performed at a size that can be calculated within 1/FPS, the maximum number of volume pixels is determined by FPS specifically. Therefore, when the pixel numbers on the X axis, the Y axis, and the Z axis in the three-dimensional space are Xn, Yn, and Zn, respectively, a relationship of Xn×Yn×Zn=<MVS is established. When the actual scales per pixel of the X axis, the Y axis, and the Z axis are Xp, Yp, and Zp, respectively, Xp=Yp=depth/(Xn or Yn) and Zp=α×Xp=α×Yp are satisfied. Although α is basically 1, when the three-dimensional image is actually constructed, a three-dimensional image that is not suitable for the image of the operator may be completed. In such a case, it is possible to construct the three-dimensional image close to a clinical cardiac cavity or a blood vessel image by adjusting a.

The actual scale to be three-dimensionally converted may be automatically determined from the relationship of Xn, Yn, depth, and FPS, whereas when the operator tries to set a pull-back distance, Zn corresponding to the distance may exceed Zm. In this case, Xn, Yn, Zn, Xp, Yp, and Zp need to be modified again.

In this manner, the meaning of each of the pixels on the X axis, the Y axis, and the Z axis is correlated with the reality, so that it is possible to construct the three-dimensional image with more reality. By separately providing the a value, it is possible to construct an actual image of the inside of the cardiac cavity imaged by the doctor. According to the present embodiment, it is possible to construct the three-dimensional image imitating the actual scale while updating the three-dimensional image in real time.

As described below, as a modification of the present embodiment, the control unit 41 may interpolate an image between the generated two-dimensional images when the moving distance Md of the ultrasound transducer 25 at each time interval Tx (=1/FPS) at which the two-dimensional images are generated is larger than the determined setting ratio Zp. That is, the control unit 41 may interpolate the image between the generated two-dimensional images when the moving distance of the ultrasound transducer 25 per unit time is larger than the product of the number FPS of the two-dimensional images generated per unit time and the determined setting ratio Zp. That is, when the scanner unit 31 moves at a relatively high speed, image interpolation may be performed.

The relationship between a linear scale of a range in which the scanner unit 31 can move and a scale on the Z axis in the three-dimensional space is defined by Z=f(z). When a moving distance at the time interval Tx is larger than a range of one pixel on the Z axis in the three-dimensional space defined by Z=f(z), a region without information is generated. That is, a speed at which the IVUS catheter can acquire the images is fixed, and when the scanner unit 31 is moved at a relatively high speed, the distance between the generated images may be significantly large. In such a case, it is necessary to perform the interpolation processing on a missing region between the images. An interpolation number needs to be changed in accordance with the relationship between the moving distance at each time interval Tx of the ultrasound transducer 25 and Z=f(z).

The interpolation processing is preferably performed by a machine learning approach, and high-speed processing can be performed by collectively performing classification for each two-dimensional image and catheter extraction processing. Each processing can be separated from each other, and each processing can be combined. Each processing is executed in parallel or in a permutation, and when the processing is executed in parallel, it is possible to achieve temporal saving.

When a range in which the three-dimensional image is updated is relatively large, it is necessary to cause the ultrasound transducer 25 to reciprocate at a higher speed in order to improve the real time property, and the range in which the image interpolation needs to be performed is relatively large. That is, a pull-back speed may be variable depending on the three-dimensional conversion range. In this case, an interpolation range also needs to be variable depending on the speed. In the IVUS, a person may freely move an imaging range by a manual pull-back method. However, in this case, it is necessary to perform an interpolation operation while constantly changing a region to be interpolated.

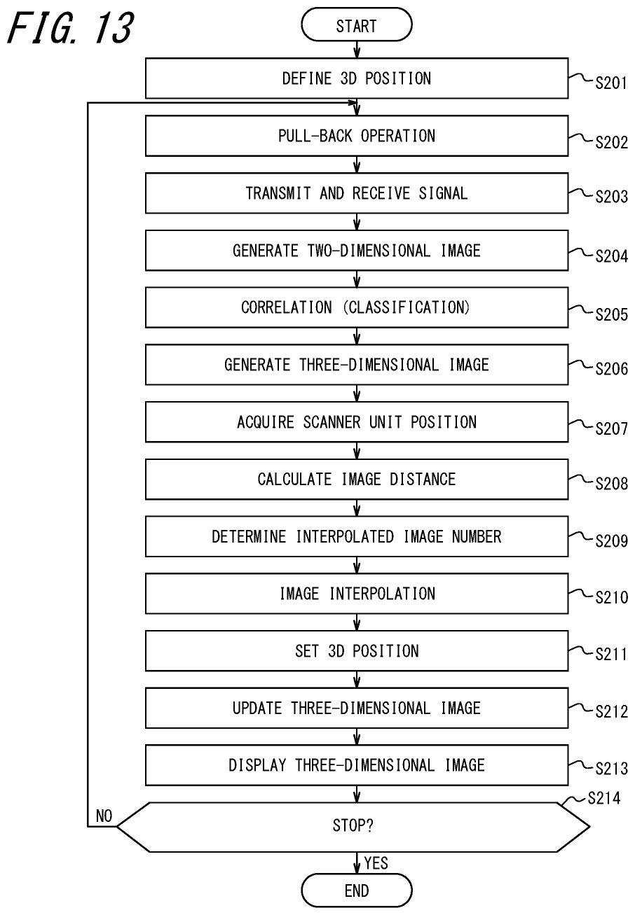

The operation of the diagnostic assistance system 10 according to the modification will be described with reference to FIG. 13.

In step S201, the control unit 41 of the diagnostic assistance device 11 defines a position in the three-dimensional space by correlating with the position of the scanner unit 31 in the pull-back operation.

The processing from step S202 to step S206 is the same as the processing from step S1 to step S5 in FIG. 5, and thus the description of step S202 to step S206 will be omitted.

In step S207, the control unit 41 of the diagnostic assistance device 11 acquires position information on the scanner unit 31 in the pull-back operation in step S202.

In step S208, the control unit 41 of the diagnostic assistance device 11 specifies the correlated position in the three-dimensional space in step S201 as a position indicated by the position information acquired in step S207. The control unit 41 calculates a distance between the specified position and the position specified in previous step S208. When the control unit 41 performs the processing in step S208 for the first time, the control unit 41 performs only the specification of the position without the calculation of the distance, and skips the processing in steps S209 to S212.

In step S209, the control unit 41 of the diagnostic assistance device 11 divides the distance calculated in step S208 by the setting ratio Zp determined in step S106 to determine an interpolated image number. That is, the control unit 41 determines the interpolated image number by dividing the moving distance of the scanner unit 31 at each time interval Tx at which the two-dimensional image is generated by the determined setting ratio Zp. When the determined interpolated image number is 0, the control unit 41 skips the processing from step S210 to step S212.

In step S210, the control unit 41 of the diagnostic assistance device 11 generates interpolated images having the number determined in step S209 using the two-dimensional image generated in step S204 and the two-dimensional image generated in previous step S204 as necessary. As a method for generating the interpolated images, a general image interpolation method may be used, or a dedicated image interpolation method may be used. An approach of the machine learning may be used.

In step S211, the control unit 41 of the diagnostic assistance device 11 sets a position to which the interpolated images generated in step S210 are applied in the three-dimensional image generated in step S206 by performing inverse calculation from the position specified in step S208 or by performing calculation from the position specified in previous step S208. For example, when the interpolated image number determined in step S209 is 1, the control unit 41 sets a position obtained by subtracting the distance corresponding to the setting ratio Zp determined in step S106 from the position specified in step S208 as a position to which the interpolated image generated in step S210 is applied. When the interpolated image number determined in step S209 is 2, the control unit 41 further sets a position obtained by subtracting the distance corresponding to twice the setting ratio Zp determined in step S106 from the position specified in step S208 as a position to which the interpolated image generated in step S210 is applied.

In step S212, the control unit 41 of the diagnostic assistance device 11 classifies a plurality of pixels included in the interpolated images generated in step S210, similarly to the processing in step S205. Then, in the processing in step S206, the control unit 41 performs the same processing as the processing in step S206 so that the two-dimensional image generated in step S204 is applied to the position specified in step S208, whereas the interpolated images generated in step S210 are applied to the position set in step S211, and generates the three-dimensional image from the classified pixel group.

The processing in step S213 and step S214 is the same as the processing in step S6 and step S7 in FIG. 5 except that the three-dimensional image generated in step S212 instead of the three-dimensional image generated in step S206 is displayed in step S213, and thus the description of step S213 and step S214 will be omitted.

The disclosure is not limited to the above-described embodiment. For example, a plurality of blocks described in the block diagram may be integrated, or one block may be divided. Instead of executing the plurality of steps described in the flowchart in time series according to the description, the steps may be executed in parallel or in a different order according to processing capability of the device that executes each step or as necessary. In addition, modifications can be made without departing from the gist of the disclosure.

For example, the image processing P1, the image processing P2, and the image processing P3 shown in FIG. 6 may be executed in parallel.

The detailed description above describes embodiments of a diagnostic assistance device and a diagnostic assistance method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic assistance device that generates a three-dimensional image of a moving range of an ultrasound transducer from a two-dimensional image generated using the ultrasound transducer, the ultrasound transducer being configured to transmit ultrasound while moving inside a biological tissue through which blood passes, the diagnostic assistance device comprising:
a control unit configured to determine an upper limit of a third pixel number according to a number of the two-dimensional image generated per unit time, a first pixel number, and a second pixel number, the first pixel number being a pixel number in a first direction of the three-dimensional image corresponding to a horizontal direction of the two-dimensional image, the second pixel number being a pixel number in a second direction of the three-dimensional image corresponding to a vertical direction of the two-dimensional image, and the third pixel number being a pixel number in a third direction of the three-dimensional image corresponding to a moving direction of the ultrasound transducer.

2. The diagnostic assistance device according to claim 1, wherein
the control unit is configured to determine a product of a reference ratio and a predetermined coefficient as a setting ratio, the reference ratio being a ratio of a dimension of the three-dimensional image in the first direction to the first pixel number or a ratio of a dimension of the three-dimensional image in the second direction to the second pixel number, and the setting ratio being a ratio of a dimension of the three-dimensional image in the third direction to the third pixel number.

3. The diagnostic assistance device according to claim 2, wherein
the dimension of the three-dimensional image in the first direction is a horizontal dimension of a range in which data of the two-dimensional image is acquired, and the dimension of the three-dimensional image in the second direction is a vertical dimension of the range in which the data of the two-dimensional image is acquired.

4. The diagnostic assistance device according to claim 2, wherein
the ultrasound transducer is configured to move in accordance with movement of a scanner unit; and
the control unit sets a value obtained by dividing an upper limit of a moving distance of the scanner unit by a product of the reference ratio and the coefficient as the third pixel number.

5. The diagnostic assistance device according to claim 4, wherein
the control unit is configured to warn a user when the value obtained by dividing the upper limit of the moving distance of the scanner unit by the product of the reference ratio and the coefficient exceeds the upper limit of the determined third pixel number.

6. The diagnostic assistance device according to claim 2, wherein
the control unit is configured to determine the product of the reference ratio and the coefficient as the setting ratio, and then determine a product of the reference ratio and a coefficient after a change as a new setting ratio when the coefficient is changed by a user.

7. The diagnostic assistance device cording to claim 6, wherein
the ultrasound transducer is configured to move in accordance with movement of a scanner unit; and
when the coefficient is changed by the user, when a value obtained by dividing an upper limit of a moving distance of the scanner unit by the product of the reference ratio and the coefficient after the change exceeds the upper limit of the determined third pixel number, the control unit is configured to warn the user.

8. The diagnostic assistance device cording to claim 2, wherein
the ultrasound transducer is configured to move in accordance with movement of a scanner unit; and
when the first pixel number and the second pixel number are changed by a user after the upper limit of the third pixel number is determined, the control unit is configured to warn the user when a value obtained by dividing an upper limit of a moving distance of the scanner unit by a product of the coefficient and a ratio exceeds an upper limit of the third pixel number, the ratio being a ratio of a dimension of the three-dimensional image in the first direction to a first pixel number after a change of the first pixel number and the second pixel number by the user or a ratio of the dimension of the three-dimensional image in the second direction to a second pixel number after the change, the upper limit of the third pixel number corresponding to the number of the two-dimensional image generated per unit time, the first pixel number after the change, and the second pixel number after the change.

9. The diagnostic assistance device according to claim 2, wherein
the control unit is configured to interpolate an image between generated two-dimensional images when a moving distance of the ultrasound transducer per unit time is larger than a product of the number of the two-dimensional image generated per unit time and the determined setting ratio.

10. The diagnostic assistance device cording to claim 9, wherein
the ultrasound transducer is configured to move in accordance with movement of a scanner unit; and
the control unit is configured to determine an interpolated image number by dividing a moving distance of the scanner unit at each time interval at which the two-dimensional image is generated by the determined setting ratio.

11. A diagnostic assistance method comprising:
transmitting, by an ultrasound transducer, ultrasound while moving inside a biological tissue through which blood passes;
generating, by a diagnostic assistance device, a three-dimensional image of a moving range of the ultrasound transducer from a two-dimensional image generated by using the ultrasound transducer; and
determining, by the diagnostic assistance device, an upper limit of a third pixel number, according to the number of the two-dimensional image generated per unit time, a first pixel number, and a second pixel number, the first pixel number being a pixel number in a first direction of the three-dimensional image corresponding to a horizontal direction of the two-dimensional image, the second pixel number being a pixel number in a second direction of the three-dimensional image corresponding to a vertical direction of the two-dimensional image, the third pixel number being a pixel number in a third direction of the three-dimensional image corresponding to a moving direction of the ultrasound transducer.

12. The diagnostic assistance method according to claim 11, further comprising:
determining, by the diagnostic assistance device, a product of a reference ratio and a predetermined coefficient as a setting ratio, the reference ratio being a ratio of a dimension of the three-dimensional image in the first direction to the first pixel number or a ratio of a dimension of the three-dimensional image in the second direction to the second pixel number, and the setting ratio being a ratio of a dimension of the three-dimensional image in the third direction to the third pixel number.

13. The diagnostic assistance method according to claim 12, wherein the dimension of the three-dimensional image in the first direction is a horizontal dimension of a range in which data of the two-dimensional image is acquired, and the dimension of the three-dimensional image in the second direction is a vertical dimension of the range in which the data of the two-dimensional image is acquired.

14. The diagnostic assistance device according to claim 12, further comprising:
moving the ultrasound transducer in accordance with movement of a scanner unit; and
setting, by the diagnostic assistance device, a value obtained by dividing an upper limit of a moving distance of the scanner unit by a product of the reference ratio and the coefficient as the third pixel number.

15. The diagnostic assistance method according to claim 14, further comprising:
warning, by the diagnostic assistance device, a user when the value obtained by dividing the upper limit of the moving distance of the scanner unit by the product of the reference ratio and the coefficient exceeds the upper limit of the determined third pixel number.

16. The diagnostic assistance method according to claim 12, further comprising:
determining, by the diagnostic assistance device, the product of the reference ratio and the coefficient as the setting ratio; and
determining, by the diagnostic assistance device, a product of the reference ratio and a coefficient after a change as a new setting ratio when the coefficient is changed by a user.

17. The diagnostic assistance method according to claim 16, further comprising:
moving the ultrasound transducer in accordance with movement of a scanner unit; and
warning, by the diagnostic assistance device, the user when the coefficient is changed by the user, when a value obtained by dividing an upper limit of a moving distance of the scanner unit by the product of the reference ratio and the coefficient after the change exceeds the upper limit of the determined third pixel number.

18. The diagnostic assistance method according to claim 12, further comprising:
moving the ultrasound transducer in accordance with movement of a scanner unit;
determining, by the diagnostic assistance device, when the first pixel number and the second pixel number are changed by a user after the upper limit of the third pixel number; and
warning, by the diagnostic assistance device, when a value obtained by dividing an upper limit of a moving distance of the scanner unit by a product of the coefficient and a ratio exceeds an upper limit of the third pixel number, the ratio being a ratio of a dimension of the three-dimensional image in the first direction to a first pixel number after a change of the first pixel number and the second pixel number by the user or a ratio of the dimension of the three-dimensional image in the second direction to a second pixel number after the change, the upper limit of the third pixel number corresponding to the number of the two-dimensional image generated per unit time, the first pixel number after the change, and the second pixel number after the change.

19. The diagnostic assistance method according to claim 12, further comprising:
interpolating, by the diagnostic assistance device, an image between generated two-dimensional images when a moving distance of the ultrasound transducer per unit time is larger than a product of the number of the two-dimensional image generated per unit time and the determined setting ratio.

20. A non-transitory computer readable medium (CRM) storing computer program code executed by a computer processor that executes a process for diagnostic assistance, the process comprising:
generating a three-dimensional image of a moving range of an ultrasound transducer from a two-dimensional image generated by using the ultrasound transducer; and
determining an upper limit of a third pixel number, according to the number of the two-dimensional image generated per unit time, a first pixel number, and a second pixel number, the first pixel number being a pixel number in a first direction of the three-dimensional image corresponding to a horizontal direction of the two-dimensional image, the second pixel number being a pixel number in a second direction of the three-dimensional image corresponding to a vertical direction of the two-dimensional image, the third pixel number being a pixel number in a third direction of the three-dimensional image corresponding to a moving direction of the ultrasound transducer.
